(19) United States
(12) Patent Application Publication
(10) Pub. No.: US 2015/0150878 A1
(43) Pub. Date: Jun. 4, 2015

(54) Methods For Inhibiting Viruses By Targeting Cathepsin-L Cleavage Sites In The Viruses' Glycoproteins

(71) Applicant: The Board of Trustees of the University of Illinois, Urbana, IL (US)

(72) Inventors: Bellur S. Prabhakar, Oak Brook, IL (US); Hatem A. Elshabrawy, Giza (EG)

(21) Appl. No.: 14/390,565

(22) PCT Filed: Apr. 4, 2013
(86) PCT No.: PCT/US2013/035319

§ 371 Date: Oct. 3, 2014

Related U.S. Application Data
(60) Provisional application No. 61/620,054, filed on Apr. 4, 2012.

(51) Int. Cl. A61K 31/5375; A61K 31/403; A61K 31/53; A61K 31/18; A61K 31/4045; A61K 31/427; A61K 31/433; A61K 31/17; A61K 31/4184

(52) U.S. Cl. CPC A61K 31/5375; A61K 31/17; A61K 31/403; A61K 31/53; A61K 31/4184; A61K 31/4045; A61K 31/427; A61K 31/433; A61K 31/18

(57) ABSTRACT

The disclosure provides methods and compositions useful for inhibiting virus requiring membrane fusion for viral entry, specifically for inhibiting severe acute respiratory syndrome coronavirus (SARS-CoV), Ebola virus (EBOV), Hendra (HeV) and Nipah (NIV) viruses by targeting Cathepsin-L (CatL) cleavages sites in the viruses' glycoproteins.

This application claims the benefit of priority to U.S. Provisional Patent Application Ser. No. 61/620,054, filed Apr. 4, 2012, the disclosure of which is incorporated herein by reference in its entirety.

### STATEMENT OF GOVERNMENT INTEREST

The invention was made with government support under grant numbers UO1 A1082206 awarded by the National Institutes of Health. The government has certain rights in the invention.

### BACKGROUND

1. Field of the Disclosure

The disclosure relates to the field of virology. In particular, the disclosure relates to methods and compositions useful for inhibition of viruses that require membrane fusion for viral entry, specifically for inhibiting severe acute respiratory syndrome coronavirus (SARS-CoV), Ebola virus (EBOV), Hendra (HeV), and Nipah (NIV) viruses by targeting Cathepsin-L (CatL) cleavages sites in the viruses' glycoproteins.

2. Description of Related Art

Enveloped viruses enter the target cells by fusion of the viral envelope with the host cell membrane followed by the delivery of the viral genome to the cytoplasm. SARS-CoV, EBOV, HeV and NiV are highly infectious zoonotic viruses of this type. SARS-CoV belongs to family Coronaviridae and causes Severe Acute Respiratory Syndrome (SARS) that initially originated in the Guangdong province of China in late 2002, spread rapidly around the world along international air-travel routes, and resulted in a mortality of 10% over different parts of the world (Peiris et al., 2004, Nat Med 10: S88-97). EBOV belongs to family Filoviridae and has been identified as the causative agent of severe hemorrhagic fever with human case fatality rate exceeding 90% in large outbreaks (Seah, 1978, Can Med Assoc J 118: 347-8, 50). NiV and HeV are closely related and belong to the genus Henipaviruses within the Paramyxoviridae family and were first identified as the etiologic agents responsible for an outbreak of fatal encephalitis among pig farmers in Malaysia and Singapore in 1999 with a case fatality of 40% (Chua et al., 1999, Lancet 354: 1257-9; Chua et al., 2000, Science 288: 1432-5; Selvey & Sheridan, 1995, J Travel Med 2: 275; Selvey et al., 1995, Med J Aust 162: 642-5).

SARS-CoV, EBOV, HeV and NiV viruses are enveloped viruses that critically require cathepsin L (CatL), a host intracellular lysosomal protease, for their glycoprotein processing and cleavage allowing for virus fusion and entry into the host cells (Simmons et al., 2005, Proc Natl Acad Sci USA 102:11876-81; Kaletsky et al., 2007, J Virol 81:13378-84; Pager & Dutch, 2005, J Virol 79:12714-20; Pager et al., 2006, Virology 346: 251-7). SARS and Ebola viruses infect target cells after cleavage of their fusion glycoproteins by CathL in the endocytic vesicles. The Hendra and Nipah viruses fusion (F₀) protein is translocated to the membrane and then internalized, permitting CatL mediated cleavage into F₁ and F₂ subunits, required for fusion. The processed F protein is then incorporated into the viral particle (Pager & Dutch, 2005, J Virol 79:12714-20; Pager et al., 2006, Virology 346: 251-7).

The high virulence of these viruses and the absence of effective therapeutic modalities and vaccines pose an ongoing threat to the public health. There are no effective therapies for the above fatal viruses to date. Accordingly, identifying a broad spectrum small molecule antiviral drug would be an advantageous and novel approach for inhibiting those fatal viruses.

Antiviral drugs can be broadly divided into 4 major classes. The earliest group of antiviral drugs to be defined consisted of nucleoside analogs that interfere with replication of the viral genome. This group includes the first successful antiviral, Acyclovir, which is effective against herpes virus infections and can delay HIV-1 progression (De Clercq & Field, 2006, Br J Pharmacol 147: 1-11; Broder, 2010, Antiviral Res 85: 1-18). The first antiviral drug to be approved for treating HIV, Zidovudine (AZT), is also a nucleoside analogue that blocks reverse transcriptase (De Clercq & Field, 2006, Br J Pharmacol 147: 1-11; Broder, 2010, Antiviral Res 85: 1-18). However, these nucleoside analogs, which have low affinities to cellular DNA polymerase can only target viruses like HIV and Herpes viruses that use their own polymerases for genome replication.

The second class of antivirals includes inhibitors of viral proteases, which are involved in the processing of viral protein chains for the final viral assembly and release. Since HIV viral assembly within the host requires a similar protease, considerable research has been performed to discover “protease inhibitors” to attack HIV at that phase of its life cycle. Although protease inhibitors became available in the 1990s and have proven effective, they have exhibited dramatic side effects (Flint et al., 2009, Toxicol Pathol 37: 65-77). The limitation of protease inhibitors use includes inability to target a wide range of viruses as they are highly specific in action and are encoded by only certain viruses.

A third-class of antivirals that have been investigated includes inhibitors of virus uncoating (Bishop, 1998, Intervirology 41: 261-71; Almela et al., 1991, J Virol 65: 2572-7). These agents act on virus penetration/uncoating and include Amantadine and Rimantadine which have been introduced to combat influenza. According to the US Centers for Disease Control and Prevention (CDC), 100% of seasonal H3N2 and 2009 pandemic flu samples tested have shown resistance to Amantadine which is no longer recommended for treatment of influenza (Salter et al., 2011, Intervirology 54: 305-15).

The final stage in the life cycle of a virus is the release of matured viruses from the host cell, and this step has also been targeted by a fourth class of antivirals. Two drugs named Zanamivir (Relenza) and Oseltamivir (Tamiflu), which have been recently introduced to treat influenza, prevent the release of viral particles by blocking neuraminidase found on the surface of flu viruses (Collins et al., 2008, Nature 453: 1258-61; Garcia-Sosa et al., 2008, J Chem Inf Model 48: 2074-80). However, the use of these neuraminidase inhibitors is restricted to neuraminidase-containing viruses.

Each of the above-mentioned antivirals is specific to a particular virus and can, thus, not be termed “broad spectrum”. In fact, some of these anti-virals are only effective against a narrow range within the target virus strains. For example, the CDC does not consider Tamiflu as an effective drug in treating H1N1 Seasonal Flu due to His274Tyr mutation which is currently widespread in 99.6% of all tested seasonal H1N1 strains. In fact, tests showed that 99.6% of the tested strains of seasonal H1N1 flu and 0.5% of 2009 pandemic flu were resistant to Tamiflu (Collins et al., 2008, Nature 453: 1258-61; Garcia-Sosa et al., 2008, J Chem Inf Model 48: 2074-80).

There have been several attempts to discover more effective inhibitors of SARS-CoV, EBOV, HeV and NiV. For example, some inhibitors of papain-like protease were described which inhibit live SARS CoV infection of VeroE6 cells (Ghosh et al., 2010, J Med Chem 53: 4968-79). Even if these inhibitors are proven to be effective in clinical trials, they are still directed against a specific virus. One group has described a small molecule named oxocarbazate which blocks human CatL thus inhibiting SARS-CoV and EBOV pseudotyped virus infection into human embryonic kidney 293T cells (Shah et al., 2010, Mol Pharmacol 78: 319-24). In another study, highly potent inhibitors of human CatL were identified by screening combinatorial pentapeptide amide collections (Brinker et al., 2000, Eur J Biochem 267: 5085-92). However, inhibitors of host proteases can be potentially harmful to host physiology.

Thus there exists a necessity of defining safe and effective broad spectrum small molecule antiviral drugs against these fatal viral infections.

### SUMMARY

Provided herein are methods for inhibiting a viral infection caused by a virus which requires membrane fusion for viral entry.

In a first aspect, the disclosure provides methods of inhibiting a viral infection in a mammal in need thereof, the viral infection being caused by a virus which requires membrane fusion for viral entry , the method comprising administering to the mammal an effective amount of a compound having structural formula (I):

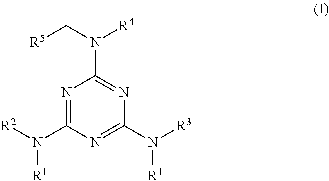

or a pharmaceutically acceptable salt thereof, wherein
each R¹ is independently hydrogen or C₁-C₆ alkyl;
R² is C₁-C₆ alkyl, C₁-C₆ haloalkyl, C₃-C₈ cycloalkyl-, heterocyclyl-, aryl-, heteroaryl-, or —C₁-C₆ alkyl-R⁶, each optionally substituted by one or more groups that are each independently halogen, cyano, nitro, hydroxy, C₁-C₆ alkoxy, amino, (C₁-C₆ alkyl)amino, or di(C₁-C₆ alkyl)amino;
R³ is C₁-C₆ alkyl, C₁-C₆ haloalkyl, C₃-C₈ cycloalkyl-, heterocyclyl-, aryl-, heteroaryl-, or —C₁-C₆ alkyl-R⁶, each optionally substituted by one or more groups that are each independently halogen, cyano, nitro, hydroxy, C₁-C₆ alkoxy, amino, (C₁-C₆ alkyl)amino, or di(C₁-C₆ alkyl)amino;
R⁴ is C₁-C₆ alkyl, C₁-C₆ haloalkyl, —CN, —C(O)C₁-C₆ alkyl, —C(O)OC₁-C₆ alkyl, —C(O)NR⁹R⁹, or —S(O)₂C₁-C₆ alkyl; and
R⁵ is hydroxy, C₁-C₆ alkoxy, amino, (C₁-C₆ alkyl)amino, di(C₁-C₆ alkyl)amino, —C(O)C₁-C₆ alkyl, —C(O)C₁-C₆ haloalkyl, —C(O)OC₁-C₆ alkyl, —C(O)OC₁-C₆ haloalkyl, or —C(O)NR⁹R⁹;
   wherein each R⁶ is independently selected from the group consisting of: —OR⁷, —SR⁷, —NR⁸R⁸, —C(O)R⁷, —C(O)OR⁷, —C(O)NR⁸R⁸, —S(O)₂NR⁸R⁸, —OC(O)R⁷, —N(R⁷)C (O)R⁷, —OC(O)OR⁷, —OC(O)NR⁸R⁸, —N(R⁷)C(O)OR⁷, —N(R⁷)C(O)NR⁸R⁸, and —N(R⁷)S(O)₂R⁷;
   and each R⁷ is independently hydrogen, C₁-C₆ alkyl, or C₁-C₆ haloalkyl;
each R⁸ is independently hydrogen or C₁-C₆ alkyl; and
each R⁹ is independently hydrogen or C₁-C₆ alkyl.

In a second aspect, the disclosure provides methods of inhibiting a viral infection in a mammal in need thereof, the viral infection being caused by a virus which requires membrane fusion for viral entry, the method comprising administering to the mammal an effective amount of a compound having the structural formula (II):

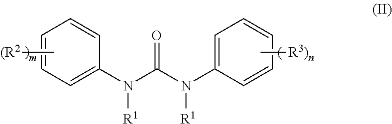

or a pharmaceutically acceptable salt thereof, wherein
m is an integer 0, 1, 2, or 3;
n is an integer 0, 1, 2, or 3;
each R¹ is independently hydrogen or C₁-C₆ alkyl;
each R² is independently halogen, —CN, —NO₂, —N₃, C₁-C₆ alkyl, —C₁-C₆ haloalkyl, —R⁴, or —C₁-C₆ alkyl-R⁴; and
each R³ is independently halogen, —CN, —NO₂, —N₃, C₁-C₆ alkyl, —C₁-C₆ haloalkyl, —R⁴, or —C₁-C₆ alkyl-R⁴,
   wherein each R⁴ is independently selected from the group consisting of: —OR⁵, —SR⁵, —S(O)R⁵, —S(O)₂R⁵, —NR⁶R⁶, —C(O)R⁵, —C(O)OR⁵, —C(O)NR⁶R⁶, —S(O)₂NR⁶R⁶, —OC(O)R⁵, —N(R⁵)C(O)R⁵, and —N(R⁵)S(O)₂R⁵, in which each R⁵ is independently hydrogen, C₁-C₆ alkyl, or C₁-C₆ haloalkyl, and each R⁶ is independently hydrogen or C₁-C₆ alkyl.

In a third aspect, the disclosure provides methods of inhibiting a viral infection a mammal in need thereof, the viral infection being caused by a virus which requires membrane fusion for viral entry, the method comprising administering to the mammal an effective amount of a compound having structural formula (III):

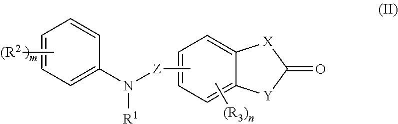

or a pharmaceutically acceptable salt thereof, wherein
X is —CH—, —NR⁷—, —O—, or —S—, where R⁷ is hydrogen or C₁-C₆ alkyl;
Y is —CH—, —NR⁸—, —O—, or —S—, where R⁸ is hydrogen or C₁-C₆ alkyl;
Z is —S(O)₂— or —C(O)—;
m is an integer 0, 1, 2, 3, or 4;
n is an integer 0, 1, 2, or 3;
each R¹ is independently hydrogen or C₁-C₆ alkyl;
each R² is independently halogen, C₁-C₆ alkyl, C₁-C₆ haloalkyl, —R⁴, or —C₁-C₆ alkyl-R⁴; and
each R³ is independently halogen, C₁-C₆ alkyl, C₁-C₆ haloalkyl, —R⁴, or —C₁-C₆ alkyl-R⁴,
   wherein each R⁴ is independently selected from the group consisting of: —CN, —NO₂, —N₃, —OR⁵, —SR⁵, —S(O)R⁵, —S(O)₂R⁵, —NR⁶R⁶, —C(O)R⁵, —C(O)OR⁵, —C(O)NR⁶R⁶, —S(O)₂NR⁶R⁶, —OC(O)R⁵, —N(R⁵)C(O)R⁵, —OC(O)OR⁵, —OC(O)NR⁶R⁶, —N(R⁵)C(O)OR⁵, —N(R⁵)C(O)NR⁶R⁶, and —N(R⁵)S(O)₂R⁵, in which each R⁵ is independently hydrogen, C₁-C₆ alkyl, or C₁-C₆ haloalkyl, and each R⁶ is independently hydrogen or C₁-C₆ alkyl.

In a fourth aspect, the disclosure provides methods of for inhibiting cathepsin L mediated cleavage of viral glycoprotein derived peptide in a virus, the method comprising contacting the virus with an effective amount of a compound having structural formula (I), structural formula (II), or structural formula (III), or a pharmaceutically acceptable salt thereof, as described herein. In certain embodiments, the virus is requires membrane fusion for viral entry. In other certain embodiments, the virus is selected from the group consisting of severe acute respiratory syndrome coronavirus, Ebola virus, Hendra virus and Nipah Virus.

In other aspects, the disclosure provides a pharmaceutical composition comprising a therapeutically effective amount of a compound having structural formula (I), (II) or (III) or a pharmaceutically acceptable salt thereof as described herein, and one or more pharmaceutically acceptable diluents, preservatives, solubilizers, emulsifiers, adjuvants, excipients, or carriers. The pharmaceutical composition can be used, for example, for inhibiting a viral infection in a mammal in need thereof, the viral infection being caused by a virus which requires membrane fusion for viral entry. In certain embodiments, the virus is selected from the group consisting of severe acute respiratory syndrome coronavirus, Ebola virus, Hendra virus and Nipah Virus.

### BRIEF DESCRIPTION OF THE DRAWINGS

FIG. 1 illustrates peptides (10 amino acids long), derived from the glycoproteins of (A) SARS-CoV, (B) Ebola GP protein derived peptide., (C) Nipah fusion protein (F₀) derived peptide, (D) Hendra fusion protein (F₀) derived peptide (E) the host pro-neuropeptide Y (pro-NPY) and (F) the host peptide F (Pep F) that contain the naturally conserved CatL cleavage sites. Arrows indicate the Cathepsin L cleavage sites in the corresponding peptides.

FIG. 2 is a MALDI-TOF mass spectrum confirming the cleavage of the synthesized SARS peptide with Cathepsin-L.

FIG. 3 is a MALDI-TOF mass spectrum confirming the cleavage of the synthesized Ebola virus peptide with Cathepsin-L.

FIG. 4 is a MALDI-TOF mass spectrum confirming the cleavage of the synthesized Nipah virus peptide with Cathepsin-L.

FIG. 5 is a MALDI-TOF mass spectrum confirming the cleavage of the synthesized Hendara virus peptide with Cathepsin-L.

FIG. 6 is a schematic of the Fluorescence Resonance Energy Transfer (FRET) based assay. In the FRET assay, if the peptide is not cleaved, no fluorescence emission is detected at 520 nm when FAM is excited at 485 nm due to the quenching effect of Tamra. In contrast, if the peptide gets cleaved, an emission of 520 nm is detected on excitation of FAM at 485 nm.

FIG. 7 is a MALDI-TOF mass spectrum confirming the cleavage of the SARS peptide labeled on the N-terminus with 5-Carboxytetramethylrhodamine (Tamra) as a quencher and on the C terminus by 5-Carboxyfluorescein (5-FAM).

FIG. 8 is a MALDI-TOF mass spectrum confirming the cleavage of the Host peptide (NPY) peptide labeled on the N-terminus with 5-Carboxytetramethylrhodamine (Tamra) as a quencher and on the C terminus by 5-Carboxyfluorescein (5-FAM).

FIG. 9 is a MALDI-TOF mass spectrum confirming the cleavage of the Ebola virus peptide labeled on the N-terminus with 5-Carboxytetramethylrhodamine (Tamra) as a quencher and on the C terminus by 5-Carboxyfluorescein (5-FAM).

FIG. 10 is a MALDI-TOF mass spectrum confirming the cleavage of the Nipah virus peptide labeled on the N-terminus with 5-Carboxytetramethylrhodamine (Tamra) as a quencher and on the C terminus by 5-Carboxyfluorescein (5-FAM).

FIG. 11 is a MALDI-TOF mass spectrum confirming the cleavage of the Hendra virus peptide labeled on the N-terminus with 5-Carboxytetramethylrhodamine (Tamra) as a quencher and on the C terminus by 5-Carboxyfluorescein (5-FAM).

FIG. 12A is a graph showing that the labeled SARS-CoV derived peptide was cleaved by CatL and the cleavage was measured in the form of increased fluorescence over time with no increase in fluorescence in CatL untreated peptide. FIG. 12B is a graph showing the cleavage rate of different viral peptides, human pro-neuropeptide Y (Pro-NPY) and peptide F (Pep F) shown as fluorescence units/min calculated from the slope of the curve at different enzyme concentrations. The bars are the average of an experiment done in triplicates and repeated twice with similar results and error bars represent SD.

FIGS. 13A-13D show the HTS screening assay statistics. FIG. 13A is a Cathepin-L replicate plot for the 5000 compounds screened against SARS peptide. FIG. 13B is a graph showing the Z-factor calculated based on the positive and negative signal obtained in the absence of the tested compounds (Z=0.061). FIG. 13C is a graph showing the positive and negative signal means per plate. FIG. 18D is a graph showing the positive and negative Standard Deviation per plate.

FIGS. 14A-14E show HTS screening assay optimization. FIGS. 14A and 14B are graphs illustrating the fluorescence emitted from a labeled viral peptide (A) SARS peptide (3 μM)+1 μg/ml Cathepsin-L enzyme (B) SARS peptide (1 μM)+0.5 μg/ml Cathepsin-L enzyme. FIG. 14C is a graph showing the fluorescence in the presence or absence of the Cathepsin-L enzyme for SARS peptide after stopping the reaction by 0.5M acetic acid (Z factor=0.69). FIGS. 14D and 14E are graphs showing the cleavage rate of different viral peptides and human peptide respectively shown as average fluorescence units/min at different enzyme concentrations.

FIG. 15 is a graph showing that the labeled SARS-CoV derived peptide (3 μM) was cleaved by CatL (50 ng) and the cleavage was measured in the form of increased fluorescence over time with no increase in fluorescence in CatL untreated peptide (3 μM).

FIG. 16 is a graph showing that the labeled SARS-CoV derived peptide (1 μM) was cleaved by CatL (100 ng and 25 ng) and the cleavage was measured in the form of increased fluorescence over time with no increase in fluorescence in CatL untreated peptide (1 μM).

FIG. 17 is a graph showing the CatL activity against labeled SARS peptide at different enzyme concentrations.

FIG. 18 is a graph illustrating the cleavage profile of SARS peptide with CatL at varying enzyme concentrations.

FIG. 19 is a graph showing the entry inhibition of EBOV, and SARS-CoV pseudotyped viruses using selected compounds. Luciferase expression was determined 72 hrs post-transduction and percentage inhibitions were calculated. VSVG pseudotyped virus and DMSO treated viruses were used as negative controls and cathepsin L (Cat L) inhibitor treated cells as a positive control. Error bars represent SD of a representative experiment performed in triplicates.

FIGS. 20A-20D are compounds that inhibit the cleavage of the SARS, Nipah, Hendra and Ebola derived peptides with significantly lower inhibition of NPY derived peptide.

FIG. 21A shows the dose dependent cleavage of recombinant SARS-CoV S-flag protein by CatL. The SARS-CoV S-flag protein was incubated with increasing concentrations of CatL (0.5, 1, and 2 μg/ml) for 4 hrs and the cleavage of the protein was detected by western blot using antiflag mouse monoclonal antibody. FIGS. 21B and 21C show the inhibition of the cat L mediated leavage of SARS-CoV S-flag protein by the compounds 5705213 and 7402683 respectively. The SARS-CoV S-flag protein was incubated with 2 μg/ml of CatL for 4 hrs at room temperature in absence or in presence of increasing concentration of the inhibitors (10-320 μM). The control (Ctrl) lane represents the S-flag protein in absence of the enzyme and inhibitor. Cat L inhibitor cat L inh.) was used as a positive control of inhibition. Asterisks represent non-specific bands.

FIGS. 22A and 22B show endogenous processing of Nipah and Hendra F₀ protein respectively in the presence and in the absence of the inhibitor 5705213 and the derivative 7402683. 293FT cells were transfected with Nipah or Hendra F₀ plasmid then treated with the inhibitors at 100 μM concentration 4 hrs post-transfection. The cathepsin L inhibitor (CatL inh.) was used as a control. The cells were lysed 48 hrs later and F₀ processing was determined by western blot using cross reactive anti-Nipah and Hendra F protein monoclonal antibody. F₀ represents uncleaved fusion protein while F₁ is the fusion subunit of the F₀ protein. FIG. 22C shows the percentage entry inhibition using various inhibitors. Nipah and Hendra pseudotyped viruses were prepared in presence of 100 μM of each inhibitor and entry into 293FT cells in presence of the inhibitors (100 μM) was quantified by measuring luciferase expression 72 hrs post-transduction. VSVG pseudotyped virus was used as a negative control and cathepsin L inhibitor (Cat L inh.) as a positive control. Error bars are SD of a representative experiment performed in triplicates.

FIGS. 23A-23D are Lineweaver-Burk plots. Different concentrations (2-64 μM) of labeled SARS-CoV derived peptide (A), labeled Ebola derived peptide (B), labeled Hendra derived peptide (C), and labeled Nipah derived peptide (D) were incubated with 0.5 μg/ml of CatL for 40 min. in the presence and in the absence of the inhibitor 5705213. The reaction was stopped with 0.5M acetic acid after which the fluorescence was measured at 535 nm after excitation at 485 nm. The velocity of the reaction was calculated as fluorescence units/min. The reciprocal of substrate concentration was plotted against the reciprocal of velocity to get the Lineweaver-Burk plot. The Kₘ was calculated from the y intercept (Vmax) and slope (Kₘ/Vₘₐₓ) in absence and in presence of the inhibitor.

FIGS. 24A-24E show the chemical structures of the inhibitory compounds identified by pseudovirus inhibition assay. Four compounds (A) Compound 5182554, (B) Compound 7910528, (C) Compound 7914021, (D) Compound 5705213, and (E) 5705213 derivative (7402683) showed inhibition of both EBOV and SARS-CoV pseudotyped virus entry.

FIGS. 25A-25B demonstrate that compound 5705213 and its derivative are not cytotoxic and their actions are specific. FIG. 25(A) is a graph showing that the four compounds identified in the pseudovirus entry inhibition assay, with the highest inhibitory effect on both EBOV and SARS-CoV pseudotyped viruses, were tested for their cytotoxic effect on 293FT cells. MTT assay was performed over 3 days using different concentrations of each compound to assess cell viability. Error bars are SD of a representative experiment performed in triplicates. FIG. 25(B) is a graph showing that different concentrations of the selected non cytotoxic compound and its derivative were tested against VSVG pseudotyped virus and the infection normalized to untreated virus was calculated. Error bars represent SD of a representative experiment performed in triplicates.

FIGS. 26A-26B are graph showing SARS-CoV pseudotyped virus entry was more inhibited by a combination of 5705213 and protease inhibitor in cells expressing TMPRSS2 protease. For FIG. 26A, entry of pseudotyped SARS-CoV was measured into 293FT cells transfected with either the human SARS-CoV receptor ACE2 plasmid only or the receptor plus increasing amounts of TMPRSS2 protease plasmid. HIV/ΔE virus which does not express the SARS-CoV spike (S) protein was used as a negative control of entry. Entry was quantified by measuring the luciferase expression in cell lysates 72 hrs post-transduction in terms of Relative Light Units (RLU). Error bars are SD of a representative experiment performed in triplicates. For FIG. 26B, entry of pseudotyped SARS-CoV into 293FT cells, transfected with either the receptor only (ACE2 plasmid) or receptor plus 10 ng of TMPRSS2 plasmid, in the presence or absence (mock) of different inhibitors. Error bars are SD of a representative experiment performed in triplicates.

### DETAILED DESCRIPTION OF THE DISCLOSURE

The disclosure provides methods for inhibiting viral replication. In particular, the methods are directed to inhibiting viral replication in viruses that utilize cathepsin L (CatL) as part of their infectious process.

In particular aspects, the methods for inhibiting viral replication are directed to viruses wherein CatL is implicated in the viral process. For example, SARS-CoV, EBOV, HeV and Niv are enveloped viruses that require CatL for glycoprotein processing and cleavage for virus fusion and entry into a host cell.

CatL is an important host protease involved in processing and biosynthesis of neuropeptides like proenkephalin, proneuropeptide Y (Pro-NPY), prodynorphin and proganalin. Thus, broadly and indiscriminately blocking CatL enzymatic activity could have dramatic side effects on human health. However, the compounds described herein inhibit the cleavage of a viral glycoprotein derived peptide with minimal inhibition to the host pro-neuropetide Y derived peptide.

One aspect of the disclosure provides methods of inhibiting a viral infection in a mammal in need thereof, the viral infection being caused by a virus which requires membrane fusion for viral entry, the method comprising administering to the mammal an effective amount of a compound having structural formula (I) as described above. In certain embodiments, the virus is selected from the group consisting of severe acute respiratory syndrome coronavirus, Ebola virus, Hendra virus and Nipah Virus.

In one embodiment, the disclosure provides methods wherein each R¹ in formula (I) is hydrogen.

In certain embodiments, the disclosure provides methods as described above with any reference to structural formula (I) where R² is C₁-C₆ alkyl, C₁-C₆ haloalkyl, C₃-C₈ cycloalkyl-, heterocyclyl-, aryl- or heteroaryl-, each optionally substituted by one or more groups that are each independently halogen, cyano, nitro, hydroxy, C₁-C₆ alkoxy, amino, (C₁-C₆ alkyl)amino, or di(C₁-C₆ alkyl)amino.

In certain embodiments, the disclosure provides methods as described above with any reference to structural formula (I) where R² is C₁-C₆ alkyl, C₁-C₆ haloalkyl, C₃-C₈ cycloalkyl-, heterocyclyl-, or —C₁-C₆ alkyl-R⁶ , each optionally substituted by one or more groups that are each independently halogen, cyano, nitro, hydroxy, C₁-C₆ alkoxy, amino, (C₁-C₆ alkyl)amino, or di(C₁-C₆ alkyl)amino. In certain such embodiments, the cycloalkyl and heterocyclyl are saturated.

In certain embodiments, R² is C₁-C₆ alkyl, C₁-C₆ haloalkyl, or —C₁-C₆ alkyl-R⁶. In other embodiments, R² is C₁-C₆ alkyl or —C₁-C₆ alkyl-R⁶, wherein R⁶ is —OR⁷, —SR⁷, or —NR⁸R⁸.

In other embodiments, the disclosure provides methods as described above with any reference to structural formula (I) where R² is C₁-C₆ alkyl. In other embodiments, R² is C₂-C₆ alkyl. In other embodiments, R² is C₂-C₅ alkyl. In other embodiments, R² is C₂-C₄ alkyl. In other embodiments, R² is C₃-C₄ alkyl.

In another embodiment, the disclosure provides methods as described above with any reference to structural formula (I) where R² is ethyl, i-propyl, or t-butyl.

In certain embodiments, the disclosure provides methods as described above with any reference to structural formula (I) where R³ is C₁-C₆ alkyl, C₁-C₆ haloalkyl, C₃-C₈ cycloalkyl-, heterocyclyl-, aryl-, heteroaryl-, C₃-C₈ cycloalkyl(C₁-C₆ alkyl)-, heterocyclyl(C₁-C₆ alkyl)-, aryl(C₁-C₆ alkyl)-, or heteroaryl(C₁-C₆ alkyl)-, each optionally substituted by one or more groups that are each independently halogen, cyano, nitro, hydroxy, C₁-C₆ alkoxy, amino, (C₁-C₆ alkyl)amino, or di(C₁-C₆ alkyl)amino.

In certain embodiments, the disclosure provides methods as described above with any reference to structural formula (I) where R³ is C₁-C₆ alkyl, C₁-C₆ haloalkyl, C₃-C₈ cycloalkyl-, heterocyclyl-, or —C₁-C₆ alkyl-R⁶ , each optionally substituted by one or more groups that are each independently halogen, cyano, nitro, hydroxy, C₁-C₆ alkoxy, amino, (C₁-C₆ alkyl)amino, or di(C₁-C₆ alkyl)amino. In certain such embodiments, the cycloalkyl and heterocyclyl are saturated.

In other embodiments, R³ is C₁-C₆ alkyl, C₁-C₆ haloalkyl, or —C₁-C₆ alkyl-R⁶. In yet other embodiments, R³ is C₁-C₆ alkyl or —C₁-C₆ alkyl-R⁶, wherein R⁶ is —OR⁷, —SR⁷, or —NR⁸R⁸.

In other embodiments, the disclosure provides methods as described above with any reference to structural formula (I) where R³ is C₁-C₆ alkyl. In other embodiments, R³ is C₂-C₆ alkyl. In other embodiments, R³ is C₂-C₅ alkyl. In other embodiments, R³ is C₂-C₄ alkyl. In other embodiments, R³ is C₃-C₃ alkyl.

In another embodiment, the disclosure provides methods as described above with any reference to structural formula (I) where R³ is ethyl, i-propyl, or t-butyl.

In other embodiments, the disclosure provides methods as described above with any reference to structural formula (I) where both R² and R³ are ethyl.

In certain embodiments, the disclosure provides methods as described above with any reference to structural formula (I) where both R² and R³ are i-propyl.

In other embodiments, the disclosure provides methods as described above with any reference to structural formula (I) where both R² and R³ are t-butyl.

In certain embodiments, the disclosure provides methods as described above with any reference to structural formula (I) where R² is i-propyl or t-butyl, and R³ is ethyl. In some embodiments, R² is t-butyl, and R³ is ethyl.

In certain embodiments, the disclosure provides methods as described above with any reference to structural formula (I) where R² is i-propyl, and R³ are t-butyl.

In other embodiments, the disclosure provides methods as described above with any reference to structural formula (I) where R⁴ is hydrogen, C₁-C₆ alkyl, C₁-C₆ haloalkyl, —CN, —C(O)C₁-C₆ alkyl, or —C(O)OC₁-C₆ alkyl. In one embodiment, R⁴ is hydrogen, C₁-C₆ alkyl, C₁-C₆ haloalkyl, or —CN. In another embodiment, R⁴ is C₁-C₆ alkyl, C₁-C₆ haloalkyl, or —CN.

In certain embodiments, the disclosure provides methods as described above with any reference to structural formula (I) where R⁴ is —CN.

In other embodiments, the disclosure provides methods as described above with any reference to structural formula (I) where R⁵ is hydrogen, —CN, —C(O)C₁-C₆ alkyl, —C(O)C₁-C₆ haloalkyl, —C(O)OC₁-C₆ alkyl, —C(O)OC₁-C₆ haloalkyl, —C(O)NR⁹R⁹, or —S(O)₂C₁-C₆ alkyl. In one embodiment, R⁵ is hydrogen or —CN.

In another embodiment, R⁵ is —C(O)C₁-C₆ alkyl, —C(O)C₁-C₆ haloalkyl, —C(O)OC₁-C₆ alkyl, —C(O)OC₁-C₆ haloalkyl, —C(O)NR⁹R⁹, or —S(O)₂C₁-C₆ alkyl. In yet another embodiment, R⁵ is —C(O)C₁-C₆ alkyl, —C(O)C₁-C₆ haloalkyl, —C(O)OC₁-C₆ alkyl, or —C(O)OC₁-C₆ haloalkyl.

In certain embodiments, the disclosure provides methods as described above with any reference to structural formula (I) wherein R⁵ is —C(O)OC₁-C₆ alkyl or —C(O)OC₁-C₆ haloalkyl.

In one embodiment, R⁵ is —C(O)OC₁-C₆ alkyl. In another embodiment, R⁵ is —C(O)₂CH₃, —C(O)₂CH₂CH₃, —C(O)₂(CH₂)₂,CH₃, —C(O)₂CH(CH₃)₂, or —C(O)₂C(CH₃)₃.

In certain embodiments, the disclosure provides methods as described above with any reference to structural formula (I) wherein R⁵ is —C(O)₂CH₃.

One exemplary embodiment according to the second aspect described herein includes methods as described above with any reference to structural formula (I) wherein:
each R¹ is independently hydrogen or C₁-C₆ alkyl;
R² is C₁-C₆ alkyl, C₁-C₆ haloalkyl, C₃-C₈ cycloalkyl-, heterocyclyl-, aryl-, heteroaryl-, or —C₁-C₆ alkyl-R⁶, each optionally substituted by one or more groups that are each independently halogen, cyano, nitro, hydroxy, C₁-C₆ alkoxy, amino, (C₁-C₆ alkyl)amino, or di(C₁-C₆ alkyl)amino;
R³ is C₁-C₆ alkyl, C₁-C₆ haloalkyl, C₃-C₈ cycloalkyl-, heterocyclyl-, aryl-, heteroaryl-, or —C₁-C₆ alkyl-R⁷, each optionally substituted by one or more groups that are each independently halogen, cyano, nitro, hydroxy, C₁-C₆ alkoxy, amino, (C₁-C₆ alkyl)amino, or di(C₁-C₆ alkyl)amino;
R⁴ is C₁-C₆ alkyl, C₁-C₆ haloalkyl, or —CN; and
R⁵ is hydroxy, C₁-C₆ alkoxy, amino, (C₁-C₆ alkyl)amino, di(C₁-C₆ alkyl)amino, —C(O)C₁-C₆ alkyl, —C(O)C₁-C₆ haloalkyl, —C(O)OC₁-C₆ alkyl, —C(O)OC₁-C₆ haloalkyl, or —C(O)NR⁹R⁹.

Another exemplary embodiment according to the second aspect described herein includes methods as described above with any reference to structural formula (I) wherein:
each R¹ is independently hydrogen or C₁-C₆ alkyl;
R² is C₁-C₆ alkyl, C₁-C₆ haloalkyl, or —C₁-C₆ alkyl-R⁶, each optionally substituted by one or more groups that are each independently halogen, cyano, nitro, hydroxy, C₁-C₆ alkoxy, amino, (C₁-C₆ alkyl)amino, or di(C₁-C₆ alkyl)amino;
R³ is C₁-C₆ alkyl, C₁-C₆ haloalkyl, or —C₁-C₆ alkyl-R⁷, each optionally substituted by one or more groups that are each independently halogen, cyano, nitro, hydroxy, C₁-C₆ alkoxy, amino, (C₁-C₆ alkyl)amino, or di(C₁-C₆ alkyl)amino;
R⁴ is C₁-C₆ alkyl, C₁-C₆ haloalkyl, or —CN; and
R⁵ is hydroxy, C₁-C₆ alkoxy, amino, (C₁-C₆ alkyl)amino, di(C₁-C₆ alkyl)amino, —C(O)C₁-C₆ alkyl, —C(O)C₁-C₆ haloalkyl, —C(O)OC₁-C₆ alkyl, —C(O)OC₁-C₆ haloalkyl, or —C(O)NR⁹R⁹.

Another exemplary embodiment according to the second aspect described herein includes methods as described above with any reference to structural formula (I) wherein:
each R¹ is independently hydrogen;
R² is C₁-C₆ alkyl or —C₁-C₆ alkyl-R⁶;
R³ is C₁-C₆ alkyl or —C₁-C₆ alkyl-R⁶;
R⁴ is C₁-C₆ alkyl, C₁-C₆ haloalkyl, or —CN; and
R⁵ is —C(O)C₁-C₆ alkyl, —C(O)C₁-C₆ haloalkyl, —C(O)OC₁-C₆ alkyl, or —C(O)OC₁-C₆ haloalkyl.

Another exemplary embodiment according to the second aspect described herein includes methods as described above with any reference to structural formula (I) wherein:
each R¹ is independently hydrogen;
R² is C₂-C₆ alkyl;
R³ is C₂-C₆ alkyl;
R⁴ is C₁-C₆ alkyl, C₁-C₆ haloalkyl, or —CN; and
R⁵ is —C(O)C₁-C₆ alkyl, —C(O)C₁-C₆ haloalkyl, —C(O)OC₁-C₆ alkyl, or —C(O)C₁-C₆ haloalkyl.

Another exemplary embodiment according to the second aspect described herein includes methods as described above with any reference to structural formula (I) wherein:
each R¹ is independently hydrogen;
R² is C₂-C₄ alkyl;
R³ is C₂-C₄ alkyl;
R⁴ is C₁-C₆ alkyl, C₁-C₆ haloalkyl, or —CN; and
R⁵ is —C(O)C₁-C₆ alkyl, —C(O)C₁-C₆ haloalkyl, —C(O)OC₁-C₆ alkyl, or —C(O)OC₁-C₆ haloalkyl.

In another embodiment, the disclosure provides methods as described above with any reference to structural formula (I) where each C₁-C₆ alkyl is a C₁-C₄ alkyl. In another embodiment, the disclosure provides methods as described above with any reference to structural formula (I) where each C₁-C₆ alkyl is a C₁-C₂ alkyl. In another embodiment, the disclosure provides methods as described above with any reference to structural formula (I) where each C₁-C₆ haloalkyl is a C₁-C₄ haloalkyl. In another embodiment, the disclosure provides methods as described above with any reference to structural formula (I) where each C₁-C₆ haloalkyl is a C₁-C₂ haloalkyl. In another embodiment, the disclosure provides methods as described above with any reference to structural formula (I) where each C₁-C₆ haloalkyl is a C₁-C₄ fluoroalkyl. In another embodiment, the disclosure provides methods as described above with any reference to structural formula (I) where each C₁-C₆ haloalkyl is a C₁-C₂ fluoroalkyl. In another embodiment, the disclosure provides methods as described above with any reference to structural formula (I) where each aryl, heteroaryl, cycloalkyl and heterocycloalkyl is monocyclic and is not fused to another ring.

Certain exemplary compounds having structural formula (I) include:

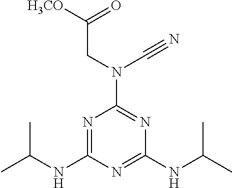 [SMILES: COC(=O)CN(C#N)c1nc(NC(C)C)nc(NC(C)C)n1]

### methyl 2-(N-(4,6-bis(isopropylamino)-1,3,5-triazin-2-yl)cyanamido)acetate; and

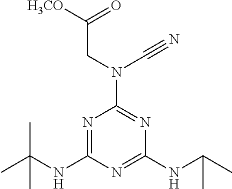 [SMILES: COC(=O)CN(C#N)c1nc(NC(C)C)nc(NC(C)(C)C)n1]

### methyl 2-(N-(4-(tert-butylamino)-6-(ethylamino)-1,3,5-triazin-2-yl)cyanamido)acetate.

These compounds can be purchased, for example, from ChemBridge Corporation.

A second aspect of the disclosure provides methods of inhibiting a viral infection in a mammal in need thereof, the viral infection being caused by a virus which requires membrane fusion for viral entry, the method comprising administering to the mammal an effective amount of a compound having the structural formula (II) as described above. In certain embodiments, the virus is selected from the group consisting of severe acute respiratory syndrome coronavirus, Ebola virus, Hendra virus and Nipah Virus.

In one embodiment, the disclosure provides methods wherein each R¹ in structural formula (II) is hydrogen.

In another embodiment, the disclosure provides methods as described above with any reference to structural formula (II) where m is an integer 1, 2, or 3. In certain embodiments, m is an integer 2 or 3. In other embodiments, m is an integer 2.

In another embodiment, the disclosure provides methods as described above with any reference to structural formula (II) where n is an integer 1, 2, or 3. In certain embodiments, n is an integer 1 or 2. In other embodiments, n is an integer 1. In still other embodiments, n is 0.

In other embodiments, the disclosure provides methods as described above with any reference to structural formula (II) where each R² and each R³ is independently halogen, —CN, —NO₂, —N₃, C₁-C₆ alkyl, C₁-C₆ haloalkyl, or —R⁴. In one embodiment, each R² and each R³ is independently halogen, —CN, —NO₂, —N₃, C₁-C₆ alkyl, or C₁-C₆ haloalkyl. In certain embodiments, each R² and each R³ is independently halogen, —CN, —NO₂, C₁-C₆ alkyl, or C₁-C₆ haloalkyl.

In other embodiments, the disclosure provides methods as described above with any reference to structural formula (II) where each R² independently halogen, —CN, —NO₂, —N₃, C₁-C₆ alkyl, C₁-C₆ haloalkyl, or —R⁴, and each R³ independently —CN, —NO₂, —N₃, C₁-C₆ alkyl, C₁-C₆ haloalkyl, or —R⁴. In one embodiment, each R² is independently halogen, —CN, —NO₂, —N₃, C₁-C₆ alkyl, or C₁-C₆ haloalkyl, and each each R³ is independently —CN, —NO₂, —N₃, C₁-C₆ alkyl, or C₁-C₆ haloalkyl. In certain embodiments, each R² is independently halogen, —CN, —NO₂, C₁-C₆ alkyl, or C₁-C₆ haloalkyl, and each R³ is independently —CN, —NO₂, C₁-C₆ alkyl, or C₁-C₆ haloalkyl.

In certain embodiments, the disclosure provides methods as described above with any reference to structural formula (II) where each R⁴ is independently selected from the group consisting of: OR⁵, —SR⁵, —S(O)R⁵, —S(O)₂R⁵, —NR⁶R⁶, C(O)R⁵, —C(O)OR⁵, —C(O)NR⁶R⁶, —S(O)₂NR⁶R⁶, —OC(O)R⁵, —N(R⁵)C(O)R⁵ and —N(R⁵)S(O)₂R⁵. In certain embodiments, the disclosure provides methods as described above with any reference to structural formula (II) where each R⁴ is independently selected from the group consisting of —OR⁵, —SR⁵, —S(O)R⁵, —S(O)₂R⁵, —NR⁶R⁶ and —C(O)R⁵.

In certain embodiments, the disclosure provides methods as described above with any reference to structural formula (II) where each R² and each R³ is independently halogen, —CN, —NO₂, C₁-C₆ haloalkyl, or —R⁴, where R⁴ is independently selected from the group consisting of: —C(O)R⁵, —C(O)OR⁵, —C(O)NR⁶R⁶, and —S(O)₂NR⁶R⁶.

In other embodiments, the disclosure provides methods as described above with any reference to structural formula (II) where each R² and each R³ is independently halogen, —CN, or C₁-C₆ haloalkyl.

In other embodiments, the disclosure provides methods as described above with any reference to structural formula (II) where each R² is independently halogen, and each R³ is C₁-C₆ haloalkyl. Optionally, in this embodiment m may be an integer 2 and n may be an integer 1.

In another embodiment, the disclosure provides methods as described above with any reference to structural formula (II) where two R² moieties represent 3,4-dihalo-substitution on the phenyl ring. Such substitution includes, but is not limited to 3,4-dichloro-; 3-chloro-4-fluoro-; 3-fluoro-4-chloro-; and 3,4-difluoro-substitution.

In another embodiment, the disclosure provides methods as described above with any reference to structural formula (II) where one R³ moiety represents 3-haloalkyl-substituted phenyl ring. Exemplary 3-haloalkyl-substitution includes 3-trifluoromethyl (e.g., one R¹ is present at 3-position)

In other embodiments, the disclosure provides methods as described above with any reference to structural formula (II) where each R² is independently halogen, and each R³ is —CN. Optionally, in this embodiment m may be an integer 1 and n may be an integer 1.

In another embodiment, the disclosure provides methods as described above with any reference to structural formula (II) where m is an integer 1, 2, or 3, and n is an integer 0 (e.g., there is no R³ substitution).

In another embodiment, the disclosure provides methods as described above with any reference to structural formula (II) where m is an integer 0 (e.g., there is no R² substitution), and n is an integer 1, 2, or 3.

In another embodiment, the disclosure provides methods as described above with any reference to structural formula (II) where m is an integer 0 (e.g., there is no R² substitution).

In another embodiment, the disclosure provides methods as described above with any reference to structural formula (II) where n is an integer 0 (e.g., there is no R³ substitution).

One exemplary embodiment according to the first aspect described herein also includes methods as described above with any reference to structural formula (II) wherein:
m is an integer 0, 1, 2, or 3;
n is an integer 0, 1, 2, or 3;
each R¹ is independently hydrogen or C₁-C₆ alkyl;
each R² is independently halogen, —CN, —NO₂, —N₃, C₁-C₆ alkyl, C₁-C₆ haloalkyl, or —R⁴; and
each R³ is independently halogen, —CN, —NO₂, —N₃, C₁-C₆ alkyl, C₁-C₆ haloalkyl, or —R⁴,
   wherein each R⁴ is independently selected from the group consisting of: —OR⁵, —SR⁵, and —NR⁶R⁶, in which each R⁵ is independently hydrogen, C₁-C₆ alkyl, or C₁-C₆ haloalkyl, and each R⁶ is independently hydrogen or C₁-C₆ alkyl.

Another exemplary embodiment according to the first aspect described herein also includes methods as described above with any reference to structural formula (II) wherein:
m is an integer 0, 1, 2, or 3;
n is an integer 0, 1, 2, or 3;
each R¹ is independently hydrogen or C₁-C₆ alkyl;
each R² is independently halogen, —CN, C₁-C₆ alkyl, or —R⁴; and
each R³ is independently halogen, —CN, C₁-C₆ alkyl, or —R⁴,
   wherein each R⁴ is independently selected from the group consisting of: —OR⁵, —SR⁵, and —NR⁶R⁶, in which each R⁵ is independently hydrogen, C₁-C₆ alkyl, or C₁-C₆ haloalkyl, and each R⁶ is independently hydrogen or C₁-C₆ alkyl.

In another embodiment, the disclosure provides methods as described above with any reference to structural formula (II) where each C₁-C₆ alkyl is a C₁-C₄ alkyl. In another embodiment, the disclosure provides methods as described above with any reference to structural formula (II) where each C₁-C₆ alkyl is a C₁-C₂ alkyl. In another embodiment, the disclosure provides methods as described above with any reference to structural formula (II) where each C₁-C₆ haloalkyl is a C₁-C₄ haloalkyl. In another embodiment, the disclosure provides methods as described above with any reference to structural formula (II) where each C₁-C₆ haloalkyl is a C₁-C₂ haloalkyl. In another embodiment, the disclosure provides methods as described above with any reference to structural formula (II) where each C₁-C₆ haloalkyl is a C₁-C₄ fluoroalkyl. In another embodiment, the disclosure provides methods as described above with any reference to structural formula (II) where each C₁-C₆ haloalkyl is a C₁-C₂ fluoroalkyl.

Exemplary compounds having structural formula (II) include:

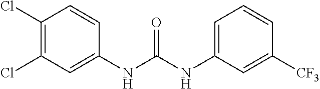 [SMILES: O=C(Nc1cccc(C(F)(F)F)c1)Nc1ccc(Cl)c(Cl)c1]

### 1-(3,4-dichlorophenyl)-3-(3-(trifluoromethyl)phenyl)urea; and

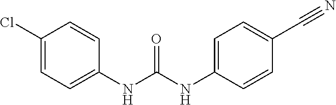 [SMILES: N#Cc1ccc(NC(=O)Nc2ccc(Cl)cc2)cc1]

### 1-(4-chlorophenyl)-3-(4-cyanophenyl)urea.

Such compounds can be purchased, for example, from ChemBridge Corporation.

A third aspect of the disclosure provides methods of inhibiting a viral infection in a mammal in need thereof, the viral infection being caused by a virus which requires membrane fusion for viral entry, the method comprising administering to the mammal an effective amount of a compound having the structural formula (III) as described above. In certain embodiments, the virus is selected from the group consisting of severe acute respiratory syndrome coronavirus, Ebola virus, Hendra virus and Nipah Virus.

In one embodiment, the disclosure provides methods wherein R¹ is hydrogen.

In other embodiments, the disclosure provides methods as described above with any reference to structural formula (III) where m is an integer 1, 2, or 3. In other embodiments, m is an integer 2 or 3. In certain embodiments, m is an integer 2.

In other embodiments, the disclosure provides methods as described above with any reference to structural formula (III) where each R² is independently halogen, —CN, —NO₂, —N₃, C₁-C₆ alkyl, C₁-C₆ haloalkyl, or —R⁴. In certain embodiments, each R² is independently halogen, —CN, —NO₂, —N₃, C₁-C₆ alkyl, or C₁-C₆ haloalkyl. In particular embodiments, each R² is independently halogen, —CN, —NO₂, —N₃, C₁-C₆ alkyl, or C₁-C₆ haloalkyl.

In certain embodiments, the disclosure provides methods as described above with any reference to structural formula (III) where wherein each R² is independently halogen, —CN, —NO₂, C₁-C₆ haloalkyl, or —R⁴, where R⁴ is independently selected from the group consisting of: —C(O)R⁵, —C(O)OR⁵, —C(O)NR⁶R⁶, and —S(O)₂NR⁶R⁶.

In other particular embodiments of the disclosure, the compounds with any reference to structural formula (III) are those wherein each R² is independently halogen, —CN, or C₁-C₆ haloalkyl.

In another embodiment, the disclosure provides methods as described above with any reference to structural formula (III) where each R² moiety is independently halogen. In certain embodiments, two R² moieties represent 3,4-dihalo-substitution on the phenyl ring. Such substitution includes, but is not limited to 3,4-dichloro-; 3-chloro-4-fluoro-; 3-fluoro-4-chloro-; and 3,4-difluoro-substitution.

In other embodiments, the disclosure provides methods as described above with any reference to structural formula (III) where n is an integer 1 or 2. In certain embodiments, n is an integer 1. In certain embodiments, n is an integer 2.

In other embodiments, the disclosure provides methods as described above with any reference to structural formula (III) wherein each R³ is independently halogen, —CN, —NO₂, —N₃, C₁-C₆ alkyl, C₁-C₆ haloalkyl, or —R⁴. In particular embodiments, each R³ is independently halogen, —CN, —NO₂, —N₃, C₁-C₆ alkyl, or C₁-C₆ haloalkyl. In other embodiments, R³ is independently halogen, —CN, C₁-C₆ alkyl, or C₁-C₆ haloalkyl. In particular embodiments, each R³ is independently halogen, —CN, or C₁-C₆ haloalkyl.

In other embodiments, the disclosure provides methods as described above with any reference to structural formula (III) where n is an integer 0 (e.g., there is no R³ substitution).

In other embodiments, the disclosure provides methods as described above with any reference to structural formula (III) where Z is —S(O)₂—.

In other embodiments, the disclosure provides methods as described above with any reference to structural formula (III) where X is —NR'-, —O—, or —S—. In particular embodiments, X is —NR¹— or —O—.

In other embodiments, the disclosure provides methods as described above with any reference to structural formula (III) where Y is —NR¹—, —O—, or —S—. In particular embodiments, Y is —NR¹— or —O—.

In particular embodiments, the disclosure provides methods as described above with any reference to structural formula (III) where one of X or Y is —NR¹—, and the other is —O—. In certain embodiments, X is —NR¹—, and Y is —O—.

In certain embodiments, the disclosure provides methods as described above with any reference to structural formula (III), having structural formula (III-a):

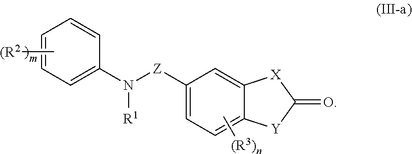

In certain embodiments, the disclosure provides methods as described above with any reference to structural formula (III), having structural formula (III-b):

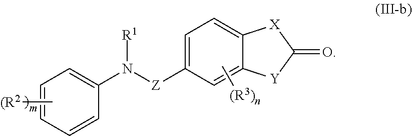

In certain embodiments, the disclosure provides methods as described above with any reference to structural formula (III), having structural formula (III-c):

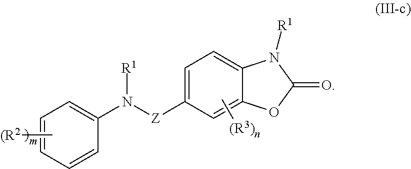

One exemplary embodiment according to the second aspect described herein includes methods as described above with any reference to structural formula (III) wherein:
X is —NR⁷—, —O—, or —S—, where R⁷ is hydrogen or C₁-C₆ alkyl;
Y is —NR⁸—, —O—, or —S—, where R⁸ is hydrogen or C₁-C₆ alkyl;
Z is —S(O)₂— or —C(O)—;
m is an integer 0, 1, 2, 3, or 4;
n is an integer 0, 1, 2, or 3;
each R¹ is independently hydrogen or C₁-C₆ alkyl;
each R² is independently halogen, —CN, —NO₂, —N₃, C₁-C₆ alkyl, C₁-C₆ haloalkyl, —R⁴, or —C₁-C₆ alkyl-R⁴; and
each R³ is independently halogen, —CN, —NO₂, —N₃, C₁-C₆ alkyl, C₁-C₆ haloalkyl, —R⁴, or —C₁-C₆ alkyl-R⁴,

Another exemplary embodiment according to the second aspect described herein includes methods as described above with any reference to structural formula (III) wherein:
X is —NR⁷—, —O—, or —S—, where R⁷ is hydrogen or C₁-C₆ alkyl;
Y is —NR⁸—, —O—, or —S—, where R⁸ is hydrogen or C₁-C₆ alkyl;
Z is —S(O)₂— or —C(O)—;
m is an integer 0, 1, 2, 3, or 4;
n is an integer 0, 1, 2, or 3;
each R¹ is independently hydrogen or C₁-C₆ alkyl;
each R² is independently halogen, —CN, —NO₂, —N₃, C₁-C₆ alkyl, C₁-C₆ haloalkyl, —R⁴, or —C₁-C₆ alkyl-R⁴; and
each R³ is independently halogen, —CN, —NO₂, —N₃, C₁-C₆ alkyl, C₁-C₆ haloalkyl, —R⁴, or —C₁-C₆ alkyl-R⁴.
   wherein each R⁴ is independently selected from the group consisting of: —OR⁵, —SR⁵, —NR⁶R⁶.

Another exemplary embodiment according to the second aspect described herein includes methods as described above with any reference to structural formula (III) wherein:
X is —NR⁷—, —O—, or —S—, where R⁷ is hydrogen or C₁-C₆ alkyl;
Y is —NR⁸—, —O—, or —S—, where R⁸ is hydrogen or C₁-C₆ alkyl;
Z is —S(O)₂— or —C(O)—;
m is an integer 0, 1, 2, or 3;
n is an integer 0, 1, or 2;
each R¹ is independently hydrogen or C₁-C₆ alkyl;
each R² is independently halogen, —CN, —NO₂, —N₃, C₁-C₆ alkyl, C₁-C₆ haloalkyl, or —R⁴; and
each R³ is independently halogen, —CN, —NO₂, —N₃, C₁-C₆ alkyl, C₁-C₆ haloalkyl, or —R⁴;
   wherein each R⁴ is independently selected from the group consisting of: —OR⁵, —SR⁵, —NR⁶R⁶.

Another exemplary embodiment according to the second aspect described herein includes methods as described above with any reference to structural formula (III) wherein:
X is —NR⁷— or —O—, where R⁷ is hydrogen or C₁-C₆ alkyl;
Y is —NR⁸— or —O—, where R⁸ is hydrogen or C₁-C₆ alkyl;
Z is —S(O)₂— or —C(O)—;
m is an integer 0, 1, 2, or 3;
n is an integer 0, 1, or 2;
each R^{l} is independently hydrogen or C₁-C₆ alkyl;
each R² is independently halogen, —CN, —NO₂, —N₃, or —R⁴; and
each R³ is independently halogen, —CN, —NO₂, —N₃, or —R⁴,
   wherein each R⁴ is independently selected from the group consisting of: —OR⁵, —SR⁵, and —NR⁶R⁶.

Another exemplary embodiment according to the second aspect described herein includes methods as described above with any reference to structural formula (III) wherein:
X is —NR⁷— or —O—, where R⁷ is hydrogen or C₁-C₆ alkyl;
Y is —NR⁸— or —O—, where R⁸ is hydrogen or C₁-C₆ alkyl;
Z is —S(O)₂—;
m is an integer 0, 1, 2, or 3;
n is an integer 0, 1, or 2;
each R¹ is independently hydrogen or C₁-C₆ alkyl;
each R² is independently halogen, —CN, —NO₂, —N₃, C₁-C₆ alkyl, C₁-C₆ haloalkyl, or —R⁴; and
each R³ is independently halogen, —CN, —NO₂, —N₃, C₁-C₆ alkyl, C₁-C₆ haloalkyl, or —R⁴,
   wherein each R⁴ is independently selected from the group consisting of: —OR⁵, —SR⁵, and —NR⁶R⁶.

In another embodiment, the disclosure provides methods as described above with any reference to structural formula (III) where each C₁-C₆ alkyl is a C₁-C₄ alkyl. In another embodiment, the disclosure provides methods as described above with any reference to structural formula (III) where each C₁-C₆ alkyl is a C₁-C₂ alkyl. In another embodiment, the disclosure provides methods as described above with any reference to structural formula (III) where each C₁-C₆ haloalkyl is a C₁-C₄ haloalkyl. In another embodiment, the disclosure provides methods as described above with any reference to structural formula (III) where each C₁-C₆ haloalkyl is a C₁-C₂ haloalkyl. In another embodiment, the disclosure provides methods as described above with any reference to structural formula (III) where each C₁-C₆ haloalkyl is a C₁-C₄ fluoroalkyl. In another embodiment, the disclosure provides methods as described above with any reference to structural formula (III) where each C₁-C₆ haloalkyl is a C₁-C₂ fluoroalkyl.

One exemplary compound having structural formula (III) is:

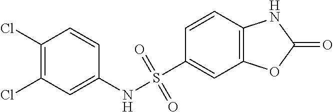 [SMILES: O=c1[nH]c2ccc(S(=O)(=O)Nc3ccc(Cl)c(Cl)c3)cc2o1]

### N-(3,4-dichlorophenyl)-2-oxo-2,3-dihydrobenzo[d]oxazole-6-sulfonamide.

This compound can, for example, be purchased from ChemBridge Corporation.

In a fourth aspect, the disclosure provides methods for inhibiting cathepsin L-mediated cleavage of viral glycoprotein-derived peptide in a virus, the method comprising contacting the virus with an effective amount of a compound having structural formula (I), structural formula (II), or structural formula (III) as described herein. As noted above and as described in more detail below, the compounds described herein can inhibit the cleavage of viral glycoprotein-derived peptide, with minimal inhibition to the host pro-neuropeptide Y-derived peptide. In certain embodiments, the virus is selected from the group consisting of severe acute respiratory syndrome coronavirus, Ebola virus, Hendra virus and Nipah Virus. But as the person of ordinary skill in the art will appreciate, the methods can be useful with any virus that utilizes cathepsin L (CatL) as part of its infectious process.

In other aspects, the disclosure provides a pharmaceutical composition comprising a therapeutically effective amount of a compound having structural formula (I), (II) or (III) as described herein, and one or more pharmaceutically acceptable diluents, preservatives, solubilizers, emulsifiers, adjuvants, excipients, or carriers. The pharmaceutical composition can be used, for example, for inhibiting a viral infection in a mammal in need thereof, the viral infection being caused by a virus selected from the group consisting of severe acute respiratory syndrome coronavirus, Ebola virus, Hendra virus and Nipah Virus.

In certain aspects, the disclosure provides a pharmaceutical composition comprising the compounds of the disclosure together with one or more pharmaceutically acceptable excipients or vehicles, and optionally other therapeutic and/or prophylactic ingredients. Such excipients include liquids such as water, saline, glycerol, polyethylene glycol, hyaluronic acid, ethanol, and the like.

The term “pharmaceutically acceptable vehicle” refers to a diluent, adjuvant, excipient or carrier with which a compound of the disclosure is administered. The terms “effective amount” or “pharmaceutically effective amount” refer to a nontoxic but sufficient amount of the agent to provide the desired biological result. That result can be reduction and/or alleviation of the signs, symptoms, or causes of a disease, or any other desired alteration of a biological system. An appropriate “effective” amount in any individual case can be determined by one of ordinary skill in the art using routine experimentation.

“Pharmaceutically acceptable carriers” for therapeutic use are well known in the pharmaceutical art, and are described, for example, in Remington's Pharmaceutical Sciences, 18th Edition (Easton, Pa.: Mack Publishing Company, 1990). For example, sterile saline and phosphate-buffered saline at physiological pH can be used. Preservatives, stabilizers, dyes and even flavoring agents can be provided in the pharmaceutical composition. For example, sodium benzoate, sorbic acid and esters of p-hydroxybenzoic acid can be added as preservatives. Id. at 1449. In addition, antioxidants and suspending agents can be used. Id.

Suitable excipients for non-liquid formulations are also known to those of skill in the art. A thorough discussion of pharmaceutically acceptable excipients and salts is available in Remington's Pharmaceutical Sciences, 18th Edition (Easton, Pa.: Mack Publishing Company, 1990).

Additionally, auxiliary substances, such as wetting or emulsifying agents, biological buffering substances, surfactants, and the like, can be present in such vehicles. A biological buffer can be any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions can be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, can include other pharmaceutical agents, adjuvants, diluents, buffers, and the like.

In general, the compositions of the disclosure will be administered in a therapeutically effective amount by any of the accepted modes of administration. Suitable dosage ranges depend upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compositions of the disclosure for a given disease.

Thus, the compositions of the disclosure can be administered as pharmaceutical formulations including those suitable for oral (including buccal and sub-lingual), rectal, nasal, topical, pulmonary, vaginal or parenteral (including intramuscular, intra-arterial, intrathecal, subcutaneous and intravenous) administration or in a form suitable for administration by inhalation or insufflation. The preferred manner of administration is intravenous or oral using a convenient daily dosage regimen which can be adjusted according to the degree of affliction.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or will be apparent, to those skilled in this art; for example, see Remington's Pharmaceutical Sciences, referenced above.

In yet another embodiment is the use of permeation enhancer excipients including polymers such as: polycations (chitosan and its quaternary ammonium derivatives, poly-L-arginine, aminated gelatin); polyanions (N-carboxymethyl chitosan, poly-acrylic acid); and, thiolated polymers (carboxymethyl cellulose-cysteine, polycarbophil-cysteine, chitosan-thiobutylamidine, chitosan-thioglycolic acid, chitosan-glutathione conjugates).

For oral administration, the composition will generally take the form of a tablet, capsule, a softgel capsule or can be an aqueous or nonaqueous solution, suspension or syrup. Tablets and capsules are preferred oral administration forms. Tablets and capsules for oral use can include one or more commonly used carriers such as lactose and corn starch. Lubricating agents, such as magnesium stearate, are also typically added. Typically, the compositions of the disclosure can be combined with an oral, non-toxic, pharmaceutically acceptable, inert carrier such as lactose, starch, sucrose, glucose, methyl callulose, magnesium stearate, dicalcium phosphate, calcium sulfate, mannitol, sorbitol and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

When liquid suspensions are used, the active agent can be combined with any oral, non-toxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, but are not limited to, preservatives, suspending agents, thickening agents, and the like.

Parenteral formulations can be prepared in conventional forms, either as liquid solutions or suspensions, solid forms suitable for solubilization or suspension in liquid prior to injection, or as emulsions. Preferably, sterile injectable suspensions are formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Parenteral administration includes intraarticular, intravenous, intramuscular, intradermal, intraperitoneal, and subcutaneous routes, and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Administration via certain parenteral routes can involve introducing the formulations of the disclosure into the body of a patient through a needle or a catheter, propelled by a sterile syringe or some other mechanical device such as an continuous infusion system. A formulation provided by the disclosure can be administered using a syringe, injector, pump, or any other device recognized in the art for parenteral administration.

Preferably, sterile injectable suspensions are formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Preparations according to the disclosure for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

Sterile injectable solutions are prepared by incorporating one or more of the compounds of the disclosure in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Thus, for example, a parenteral composition suitable for administration by injection is prepared by stirring 1.5% by weight of active ingredient in 10% by volume propylene glycol and water. The solution is made isotonic with sodium chloride and sterilized.

Alternatively, the pharmaceutical compositions of the disclosure can be administered in the form of suppositories for rectal administration. These can be prepared by mixing the agent with a suitable nonirritating excipient which is solid at room temperature but liquid at the rectal temperature and therefore will melt in the rectum to release the drug. Such materials include cocoa butter, beeswax and polyethylene glycols.

The pharmaceutical compositions of the disclosure can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, propellants such as fluorocarbons or nitrogen, and/or other conventional solubilizing or dispersing agents.

Preferred formulations for topical drug delivery are ointments and creams. Ointments are semisolid preparations which are typically based on petrolatum or other petroleum derivatives. Creams containing the selected active agent, are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

Formulations for buccal administration include tablets, lozenges, gels and the like. Alternatively, buccal administration can be effected using a transmucosal delivery system as known to those skilled in the art. The compounds of the disclosure can also be delivered through the skin or muscosal tissue using conventional transdermal drug delivery systems, i.e., transdermal “patches” wherein the agent is typically contained within a laminated structure that serves as a drug delivery device to be affixed to the body surface. In such a structure, the drug composition is typically contained in a layer, or “reservoir,” underlying an upper backing layer. The laminated device can contain a single reservoir, or it can contain multiple reservoirs. In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, can be either a polymeric matrix as described above, or it can be a liquid or gel reservoir, or can take some other form. The backing layer in these laminates, which serves as the upper surface of the device, functions as the primary structural element of the laminated structure and provides the device with much of its flexibility. The material selected for the backing layer should be substantially impermeable to the active agent and any other materials that are present.

The compositions of the disclosure can be formulated for aerosol administration, particularly to the respiratory tract and including intranasal administration. The compound will generally have a small particle size for example of the order of 5 microns or less. Such a particle size can be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol can conveniently also contain a surfactant such as lecithin. The dose of drug can be controlled by a metered valve. Alternatively the active ingredients can be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition can be presented in unit dose form for example in capsules or cartridges of e.g., gelatin or blister packs from which the powder can be administered by means of an inhaler.

A pharmaceutically or therapeutically effective amount of the composition will be delivered to the subject. The precise effective amount will vary from subject to subject and will depend upon the species, age, the subject's size and health, the nature and extent of the condition being treated, recommendations of the treating physician, and the therapeutics or combination of therapeutics selected for administration. Thus, the effective amount for a given situation can be determined by routine experimentation. For purposes of the disclosure, generally a therapeutic amount will be in the range of about 0.01 mg/kg to about 250 mg/kg body weight, more preferably about 0.1 mg/kg to about 10 mg/kg, in at least one dose. In larger mammals the indicated daily dosage can be from about 1 mg to 300 mg, one or more times per day, more preferably in the range of about 10 mg to 200 mg. The subject can be administered as many doses as is required to reduce and/or alleviate the signs, symptoms, or causes of the disorder in question, or bring about any other desired alteration of a biological system. When desired, formulations can be prepared with enteric coatings adapted for sustained or controlled release administration of the active ingredient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

### Definitions

The following terms and expressions used herein have the indicated meanings.

Terms used herein may be preceded and/or followed by a single dash, “—”, or a double dash, “═”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” unless a dash indicates otherwise. For example, C₁-C₆alkoxycarbonyloxy and —OC(O)C₁-C₆ alkyl indicate the same functionality; similarly arylalkyl and -alkylaryl indicate the same functionality.

The term “alkenyl” as used herein, means a straight or branched chain hydrocarbon containing from 2 to 10 carbons, unless otherwise specified, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl” as used herein, means a straight or branched chain hydrocarbon containing from 1 to 10 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂—, —CH₂CH₂CHC(CH₃)—, and —CH₂CH(CH₂CH₃)CH₂—.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl,” as used herein, means a phenyl (i.e., monocyclic aryl), or a bicyclic ring system containing at least one phenyl ring or an aromatic bicyclic ring containing only carbon atoms in the aromatic bicyclic ring system. The bicyclic aryl can be azulenyl, naphthyl, or a phenyl fused to a monocyclic cycloalkyl, a monocyclic cycloalkenyl, or a monocyclic heterocyclyl. The bicyclic aryl is attached to the parent molecular moiety through any carbon atom contained within the phenyl portion of the bicyclic system, or any carbon atom with the napthyl or azulenyl ring. The fused monocyclic cycloalkyl or monocyclic heterocyclyl portions of the bicyclic aryl are optionally substituted with one or two oxo and/or thia groups. Representative examples of the bicyclic aryls include, but are not limited to, azulenyl, naphthyl, dihydroinden-1-yl, dihydroinden-2-yl, dihydroinden-3-yl, dihydroinden-4-yl, 2,3-dihydroindol-4-yl, 2,3-dihydroindol-5-yl, 2,3-dihydroindol-6-yl, 2,3-dihydroindol-7-yl, inden-1-yl, inden-2-yl, inden-3-yl, inden-4-yl, dihydronaphthalen-2-yl, dihydronaphthalen-3-yl, dihydronaphthalen-4-yl, dihydronaphthalen-1-yl, 5,6,7,8-tetrahydronaphthalen-1-yl, 5,6,7,8-tetrahydronaphthalen-2-yl, 2,3-dihydrobenzofuran-4-yl, 2,3-dihydrobenzofuran-5-yl, 2,3-dihydrobenzofuran-6-yl, 2,3-dihydrobenzofuran-7-yl, benzo[d][1,3]dioxol-4-yl, benzo[d][1,3]dioxol-5-yl, 2H-chromen-2-on-5-yl, 2H-chromen-2-on-6-yl, 2H-chromen-2-on-7-yl, 2H-chromen-2-on-8-yl, isoindoline-1,3-dion-4-yl, isoindoline-1,3-dion-5-yl, inden-1-on-4-yl, inden-1-on-5-yl, inden-1-on-6-yl, inden-1-on-7-yl, 2,3-dihydrobenzo[b][1,4]dioxan-5-yl, 2,3-dihydrobenzo[b][1,4]dioxan-6-yl, 2H-benzo[b][1,4]oxazin3(4H)-on-5-yl, 2H-benzo[b][1,4]oxazin3(4H)-on-6-yl, 2H-benzo[b][1,4]oxazin3(4H)-on-7-yl, 2H-benzo[b][1,4]oxazin3(4H)-on-8-yl, benzo[d]oxazin-2(3H)-on-5-yl, benzo[d]oxazin-2(3H)-on-6-yl, benzo[d]oxazin-2(3H)-on-7-yl, benzo[d]oxazin-2(3H)-on-8-yl, quinazolin-4(3H)-on-5-yl, quinazolin-4(3H)-on-6-yl, quinazolin-4(3H)-on-7-yl, quinazolin-4(3H)-on-8-yl, quinoxalin-2(1H)-on-5-yl, quinoxalin-2(1H)-on-6-yl, quinoxalin-2(1H)-on-7-yl, quinoxalin-2(1H)-on-8-yl, benzo[d]thiazol-2(3H)-on-4-yl, benzo[d]thiazol-2(3H)-on-5-yl, benzo[d]thiazol-2(3H)-on-6-yl, and, benzo[d]thiazol-2(3H)-on-7-yl. In certain embodiments, the bicyclic aryl is (i) naphthyl or (ii) a phenyl ring fused to either a 5 or 6 membered monocyclic cycloalkyl, a 5 or 6 membered monocyclic cycloalkenyl, or a 5 or 6 membered monocyclic heterocyclyl, wherein the fused cycloalkyl, cycloalkenyl, and heterocyclyl groups are optionally substituted with one or two groups which are independently oxo or thia. In certain embodiments of the disclosure, the aryl group is phenyl or naphthyl. In certain other embodiments, the aryl group is phenyl.

The terms “cyano” and “nitrile” as used herein, mean a —CN group.

The term “cycloalkyl” as used herein, means a monocyclic or a bicyclic cycloalkyl ring system. Monocyclic ring systems are cyclic hydrocarbon groups containing from 3 to 8 carbon atoms, where such groups can be saturated or unsaturated, but not aromatic. In certain embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)₂—, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. Cycloalkyl groups are optionally substituted with one or two groups which are independently oxo or thia. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In certain embodiments of the disclosure, the cycloalkyl is cyclopentyl, cyclohexyl, or cycloheptyl,

The term “halo” or “halogen” as used herein, means —Cl, —Br, —I or —F. In certain embodiments, “halo” or “halogen” refers to —Cl or —F.

The term “haloalkyl” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl. In certain embodiments, each “haloalkyl” is a fluoroalkyl, for example, a polyfluoroalkyl such as a substantially perfluorinated alkyl.

The term “heteroaryl,” as used herein, means a monocyclic heteroaryl or a bicyclic ring system containing at least one heteroaromatic ring. The monocyclic heteroaryl can be a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. The fused cycloalkyl or heterocyclyl portion of the bicyclic heteroaryl group is optionally substituted with one or two groups which are independently oxo or thia. When the bicyclic heteroaryl contains a fused cycloalkyl, cycloalkenyl, or heterocyclyl ring, then the bicyclic heteroaryl group is connected to the parent molecular moiety through any carbon or nitrogen atom contained within the monocyclic heteroaryl portion of the bicyclic ring system. When the bicyclic heteroaryl is a monocyclic heteroaryl fused to a phenyl ring, then the bicyclic heteroaryl group is connected to the parent molecular moiety through any carbon atom or nitrogen atom within the bicyclic ring system. Representative examples of bicyclic heteroaryl include, but are not limited to, benzimidazolyl, benzofuranyl, benzothienyl, benzoxadiazolyl, benzoxathiadiazolyl, benzothiazolyl, cinnolinyl, 5,6-dihydroquinolin-2-yl, 5,6-dihydroisoquinolin-1-yl, furopyridinyl, indazolyl, indolyl, isoquinolinyl, naphthyridinyl, quinolinyl, purinyl, 5,6,7,8-tetrahydroquinolin-2-yl, 5,6,7,8-tetrahydroquinolin-3-yl, 5,6,7,8-tetrahydroquinolin-4-yl, 5,6,7,8-tetrahydroisoquinolin-1-yl, thienopyridinyl, 4,5,6,7-tetrahydrobenzo[c][1,2,5]oxadiazolyl, and 6,7-dihydrobenzo[c][1,2,5]oxadiazol-4(5H)-onyl. In certain embodiments, the fused bicyclic heteroaryl is a 5 or 6 membered monocyclic heteroaryl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused cycloalkyl, cycloalkenyl, and heterocyclyl groups are optionally substituted with one or two groups which are independently oxo or thia. In certain embodiments of the disclosure, the heteroaryl group is furyl, imidazolyl, pyridinyl, pyrimidinyl, pyrazinyl, pyrazolyl, pyrrolyl, thiazolyl, thienyl, triazolyl, benzimidazolyl, benzofuranyl, indazolyl, indolyl, or quinolinyl.

The term “heterocyclyl” as used herein, means a monocyclic heterocycle or a bicyclic heterocycle. The monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The monocyclic heterocycle is connected to the parent molecular moiety through any carbon atom or any nitrogen atom contained within the monocyclic heterocycle. Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to either a phenyl, a monocyclic cycloalkyl, a monocyclic cycloalkenyl, a monocyclic heterocycle, or a monocyclic heteroaryl. The bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. Heterocyclyl groups are optionally substituted with one or two groups which are independently oxo or thia. In certain embodiments, the bicyclic heterocyclyl is a 5 or 6 membered monocyclic heterocyclyl ring fused to phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. In certain embodiments of the disclosure, the heterocyclyl is pyrrolidinyl, piperidinyl, piperazinyl, or morpholinyl.

The term “saturated” as used herein means the referenced chemical structure does not contain any multiple carbon-carbon bonds. For example, a saturated cycloalkyl group as defined herein includes cyclohexyl, cyclopropyl, and the like.

The term “unsaturated” as used herein means the referenced chemical structure contains at least one multiple carbon-carbon bond, but is not aromatic. For example, a unsaturated cycloalkyl group as defined herein includes cyclohexenyl, cyclopentenyl, cyclohexadienyl, and the like.

“Pharmaceutically acceptable salt” refers to both acid and base addition salts.

“Modulating” or “modulate” refers to the treating, prevention, suppression, enhancement or induction of a function, condition or disorder. For example, it is believed that the compounds of the present disclosure can modulate atherosclerosis by stimulating the removal of cholesterol from atherosclerotic lesions in a human.

“Treating” or “treatment” as used herein covers the treatment of a disease or disorder described herein, in a subject, preferably a human, and includes:

i. inhibiting a disease or disorder, i.e., arresting its development;

ii. relieving a disease or disorder, i.e., causing regression of the disorder;

iii. slowing progression of the disorder; and/or

iv inhibiting, relieving, or slowing progression of one or more symptoms of the disease or disorder

“Subject” refers to a warm blooded animal such as a mammal, preferably a human, or a human child, which is afflicted with, or has the potential to be afflicted with one or more diseases and disorders described herein.

### EXAMPLES

The following Examples are illustrative of specific embodiments of the disclosure, and various uses thereof. They set forth for explanatory purposes only, and are not to be taken as limiting the disclosure.

Compounds as described herein can be prepared by chemical synthesis procedures known in the art. The person of ordinary skill will adapt known processes for making substituted urea, substituted triazines, substituted carboxamides and substituted sulfonamides to arrive at the compounds described herein. Many general references providing commonly known chemical synthetic schemes and conditions are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978). The prepared compounds can be purified by any of the means known in the art, including chromatographic means, such as HPLC, preparative thin layer chromatography, flash column chromatography and ion exchange chromatography. During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4.sup.th edition, Vol. 15/1, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups may be removed at a convenient subsequent stage using methods known from the art.

### Example 1—Cells

293FT cells were grown in Dulbecco's Modified Eagles Medium (DMEM, Cell gro) supplemented with L-Glutamine (Invitrogen), Sodium Pyruvate (Invitrogen), Non-essential amino acids (Invitrogen), and 10% Fetal Bovine Serum (FBS). The cells were used for preparation of SARS-CoV, Ebola, Hendra, Nipah, and VSVG pseudotyped viruses and for the Ebola, Hendra, Nipah, VSVG pseudotyped viruses entry inhibition experiments discussed below. The 293FT transiently transfected with the human ACE2 expression plasmid were used for the SARS-CoV pseudotyped viruses entry inhibition experiments discussed below.

### Example 2—Synthesis of Viral and Host Proteins Derived Peptides That Contain the Natural Cathepsin L Cleavage Sites

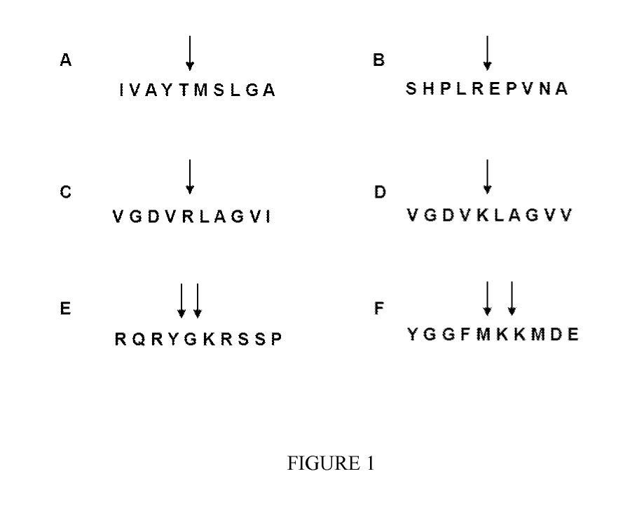

The cathepsin L (CatL) cleavage sites in the glycoproteins of SARS-CoV, EBOV, NiV and HeV zoonotic viruses were identified as conserved elements. Peptides (10 amino acids long), derived from the glycoproteins of SARS-CoV, EBOV, HeV, NiV, the host pro-neuropeptide Y (pro-NPY) and the host peptide F (Pep F) that contain the naturally conserved CatL cleavage sites, were synthesized in the protein research laboratory at UIC (FIG. 1). The peptides contained the natural cathepsin L cleavage sites in the viral proteins and host pro-NPY. The viral and host pro-NPY derived peptides were labeled on the N-terminus with 5-Carboxytetramethylrhodamine (Tamra) as a quencher and on the C terminus by 5-Carboxyfluorescein (5-FAM) as an emitter in the protein research laboratory at UIC. The labeled peptides were purified using reversed phase High performance Liquid Chromatography (HPLC) in the UIC protein research laboratory.

The cleavage products for the labeled and unlabeled peptides were analyzed by MALDI-TOF Mass Spectrometry in the UIC protein research laboratory. The SARS-CoV-S, EBOV-GP, HeV-F₀, NiV-F₀, the host pro-NPY, and Pep F derived labeled and unlabeled peptides (1 μM) were incubated with 1 μg/ml of human cathepsin L purified from human liver (Sigma Aldrich) for 1 hour at room temperature in ammonium acetate buffer pH 5.5 containing 4 mM EDTA and 8 mM dithiothreitol (DTT).

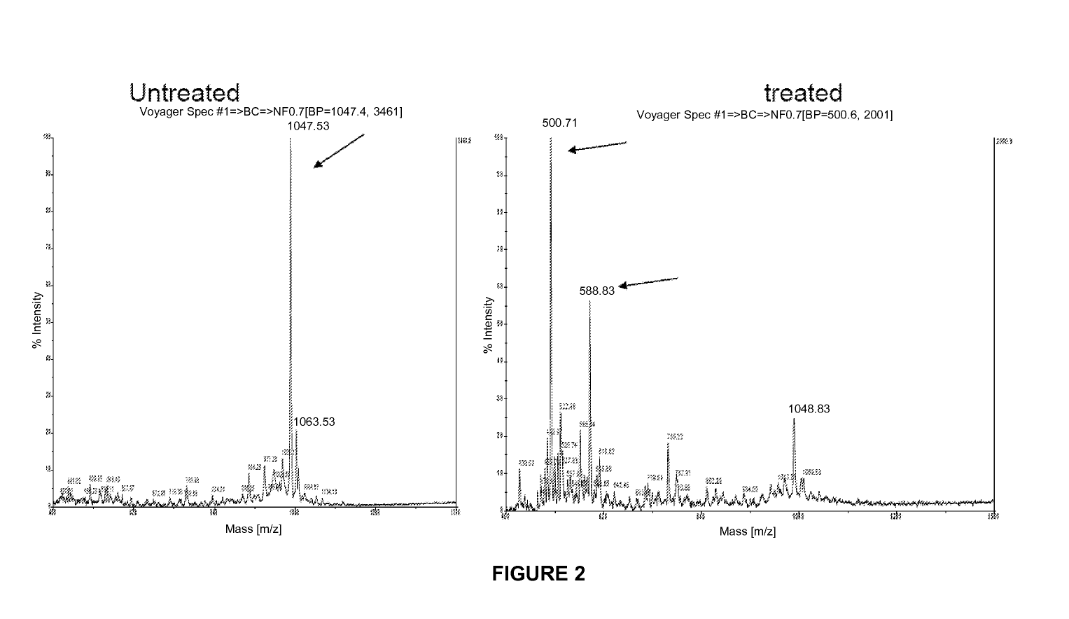

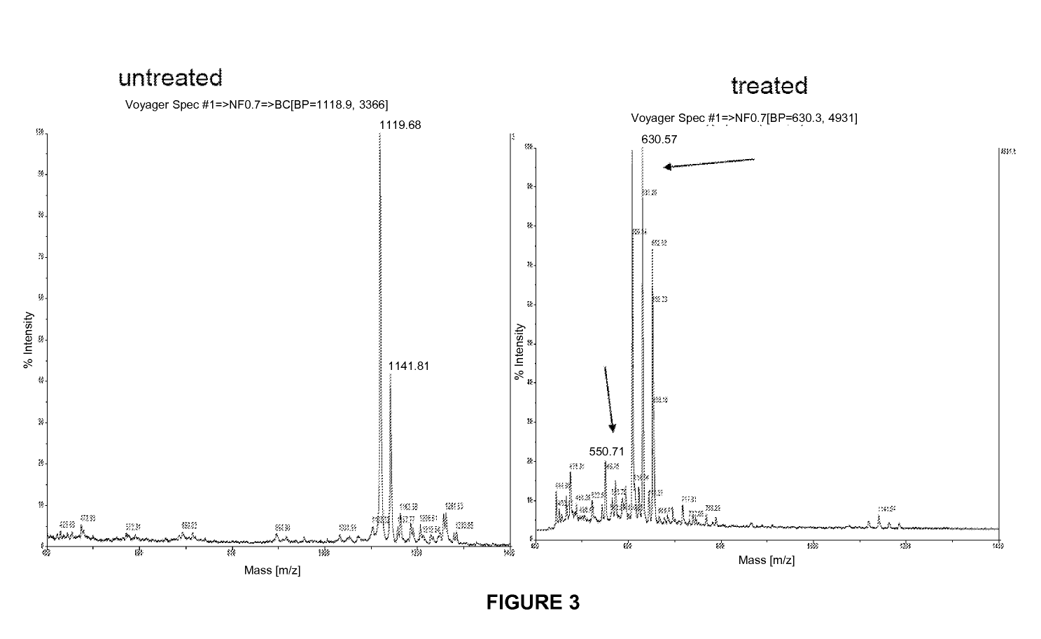

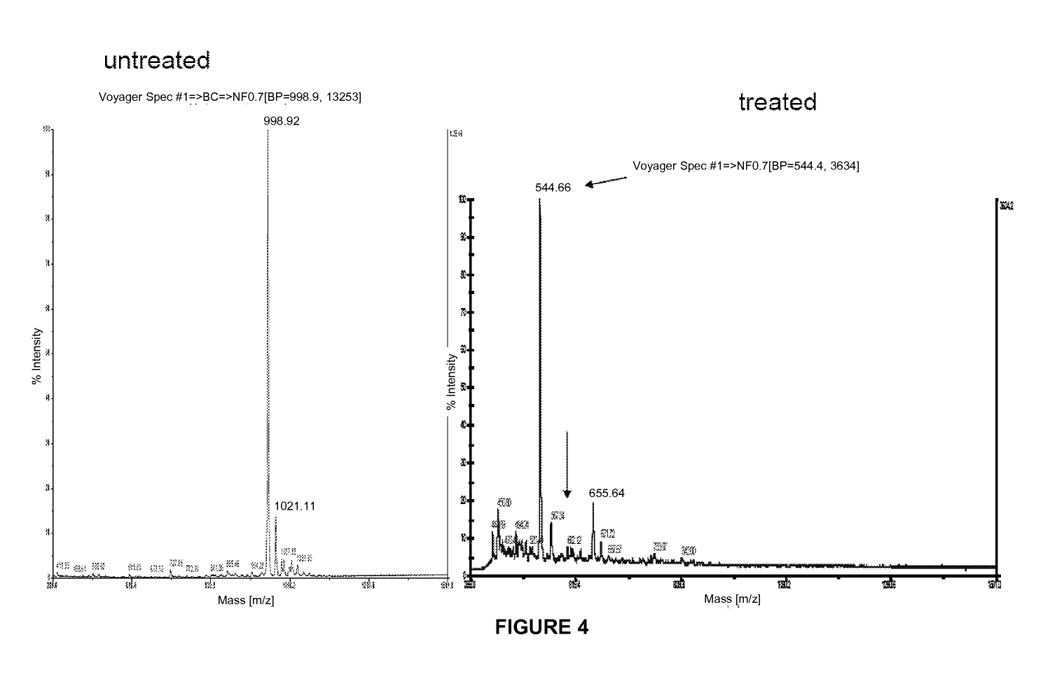

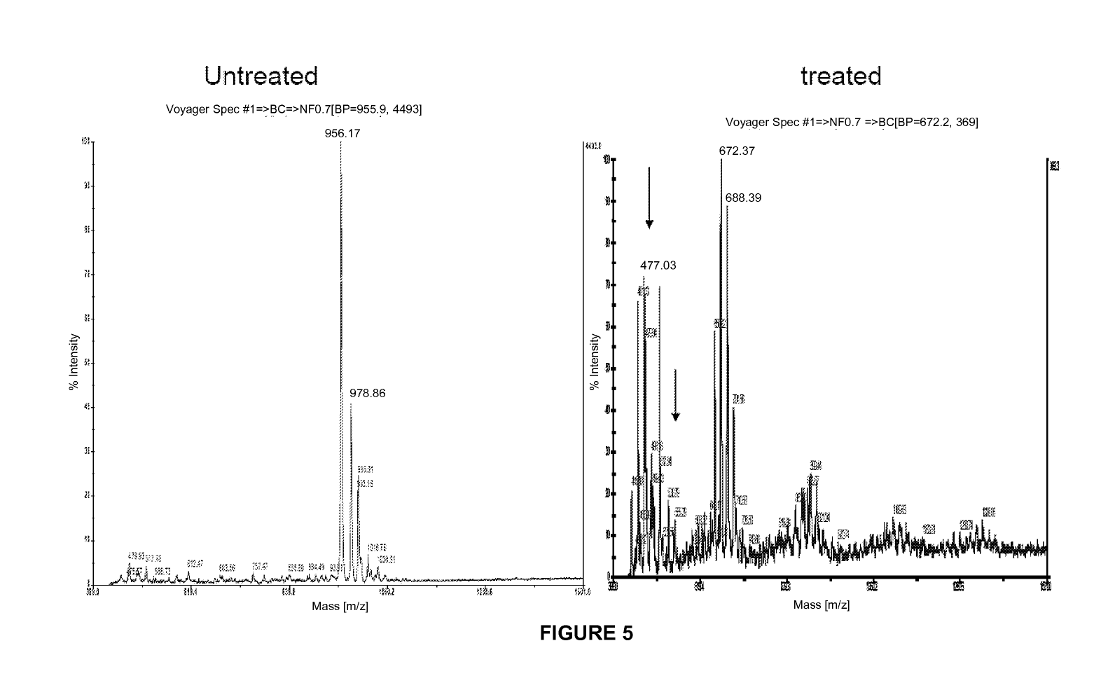

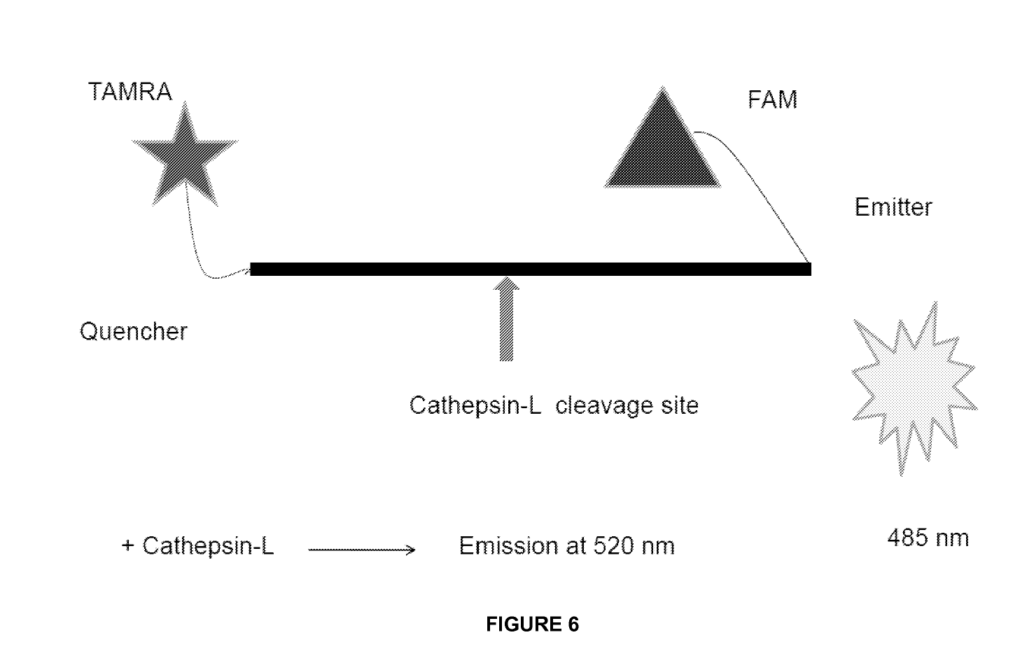

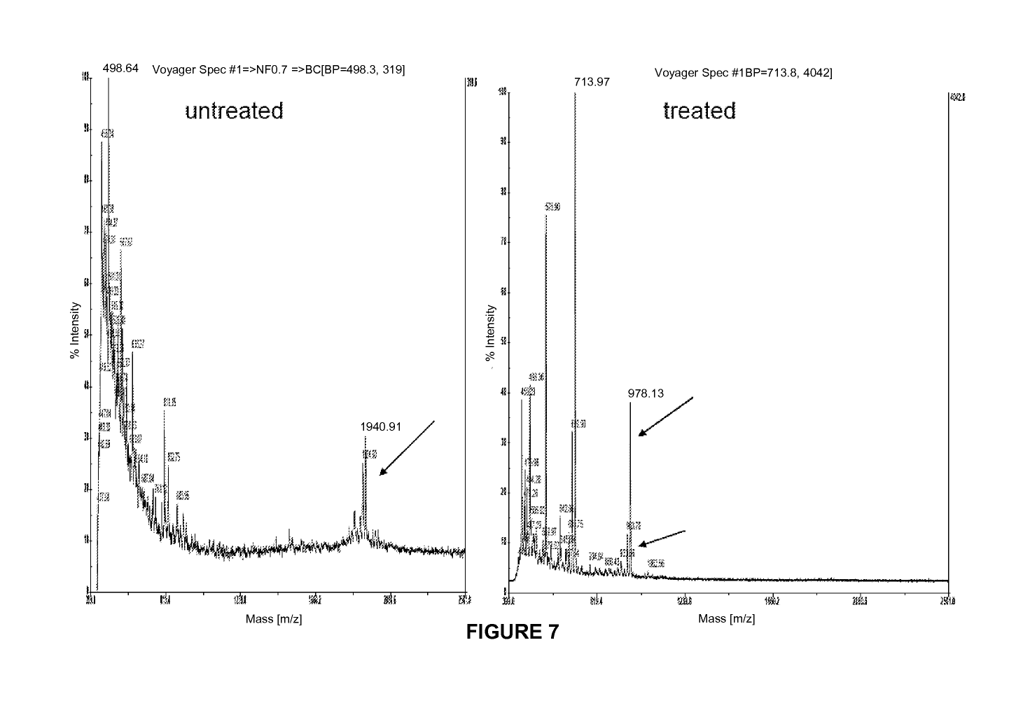

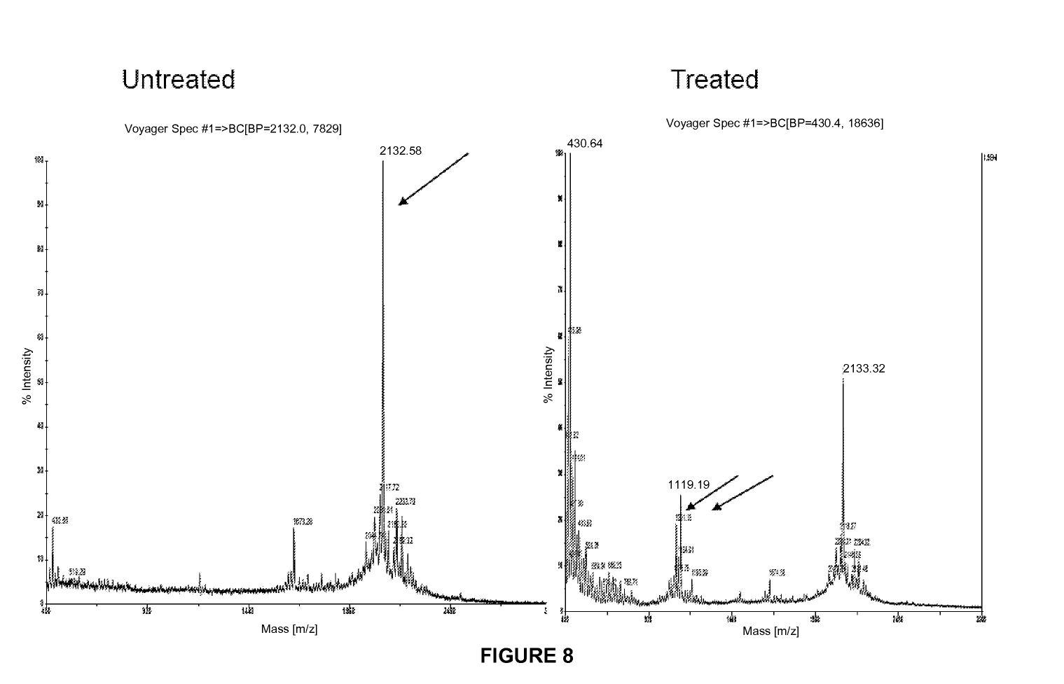

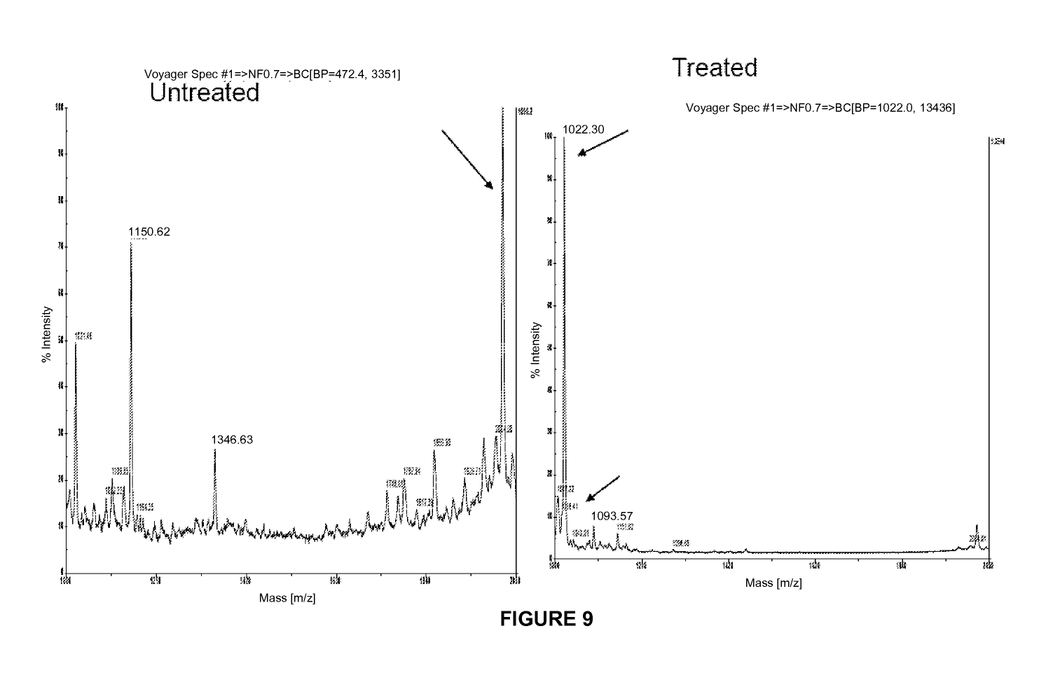

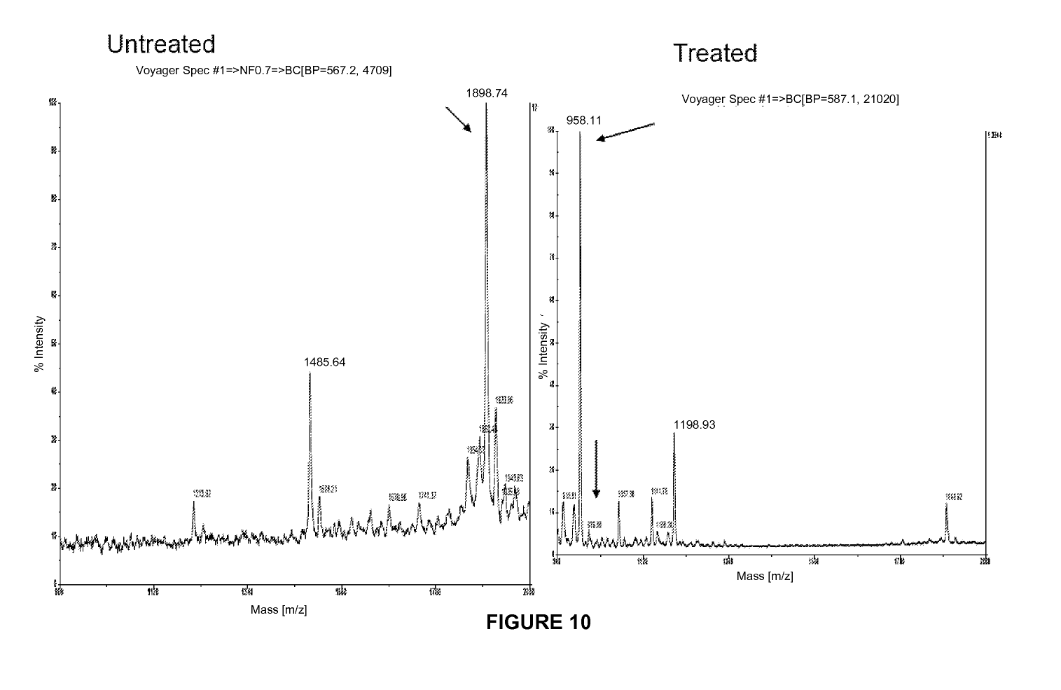

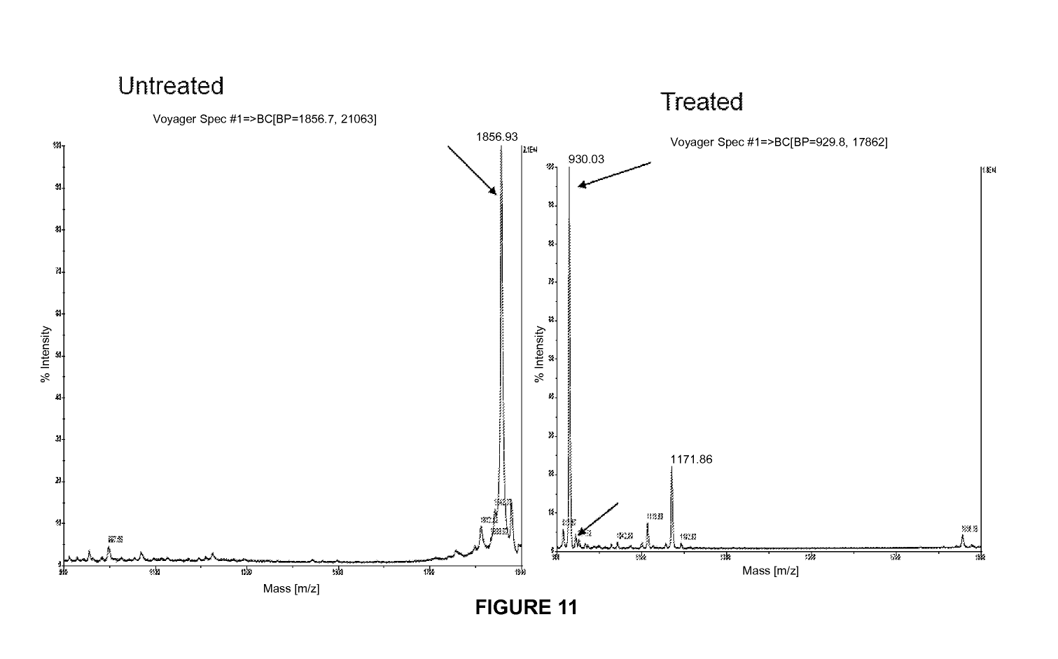

If the peptide was not cleaved, no fluorescence emission was detected at 535 nm when FAM was excited at 485 nm due to the quenching effect of Tamra. In contrast, if the peptide was cleaved by CatL, an emission of light at 535 nm was detected. See FIG. 6. The four viral derived peptides and the host derived peptides were found to be cleaved at the expected sites by Mass Spectrometry ((FIGS. 2-5). All the labeled peptides were similarly cleaved by CatL confirming that the fluorophores did not affect the CatL mediated cleavage (FIGS. 7-11).

### Example 3—Optimization of the High Throughput Screening Assay (HTSA)

The HTSA is a Fluorescence Resonance Energy Transfer (FRET) based assay. The labeled SARS-CoV S protein derived peptide was used as a substrate in the primary screen. The assay was optimized in black 384 well plates (Thermoscientific) using 3 μM SARS-CoV-S derived labeled peptide incubated with 1 μg/ml human cathepsin L (Sigma Aldrich) and further optimized with 1 μM SARS-CoV-S derived labeled peptide incubated at room temperature with 0.25, 0.5, and 1 μg/ml catL in 50 μl total volume of NH₄Ac buffer pH 5.5 supplemented with 4 mM EDTA and 8 mM DTT. The fluorescence was measured over time, at 535 nm after excitation at 485 nm, using fluorescence reader at the UIC HTS facility. The EBOV GP, HeV and NiV F₀ derived labeled peptides as well as the host pro-NPY and Pep F derived peptide were tested for cleavage concentration by incubation 1 μM of each peptide with different concentrations of cathepsin L (0.25, 0.5, and 1 μg/ml). The rate of the reaction was measured based on the slope. The quality of the screening assay (Z factor) was determined using the following formula: Z-factor=1−(3(σₚ+σₙ)/|μₚ−μₙ|), where σₚ=standard deviation of the positive signal, σₙ=standard deviation of the negative signal, μₚ=mean of the positive signal, and μₙ=mean of the negative signal. Z-Factor between 0.5 and 1 means an excellent screening assay.

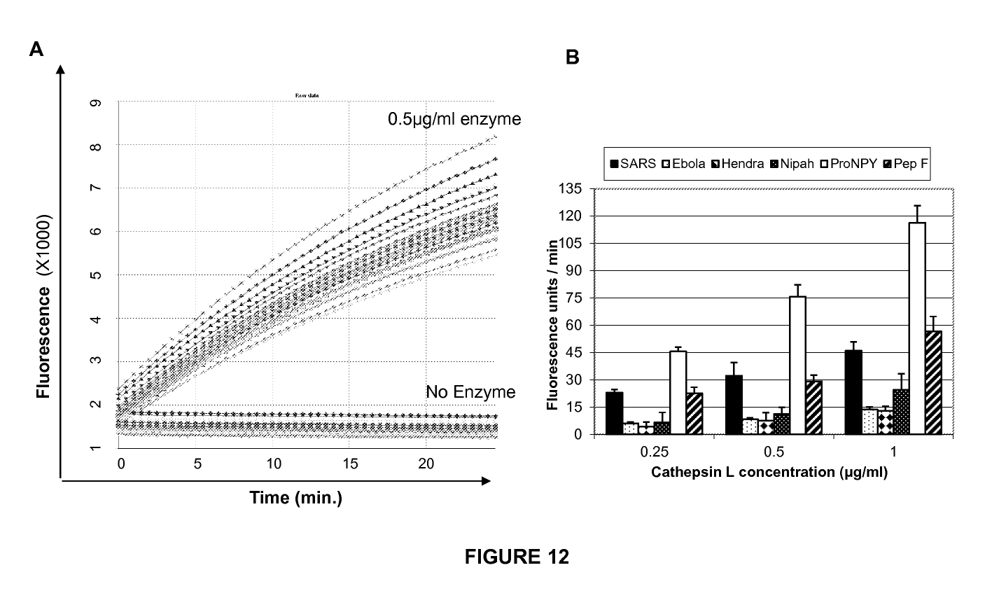

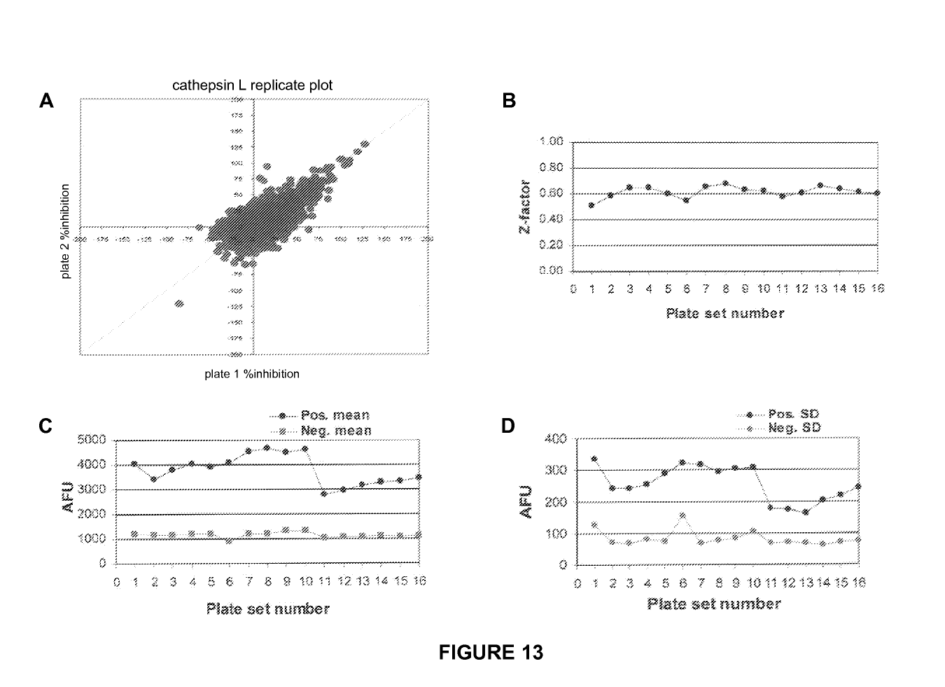

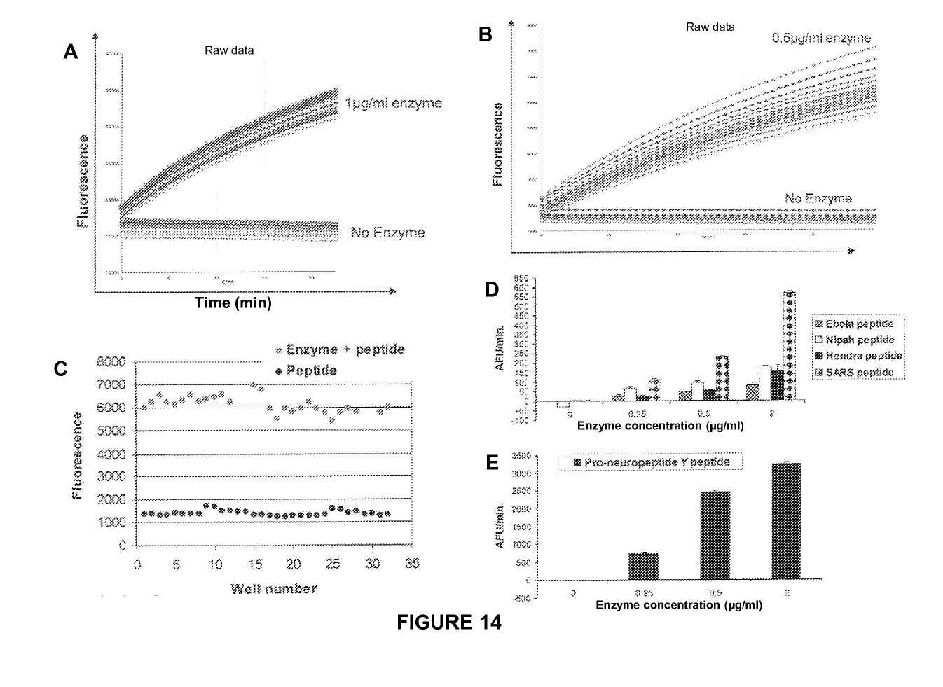

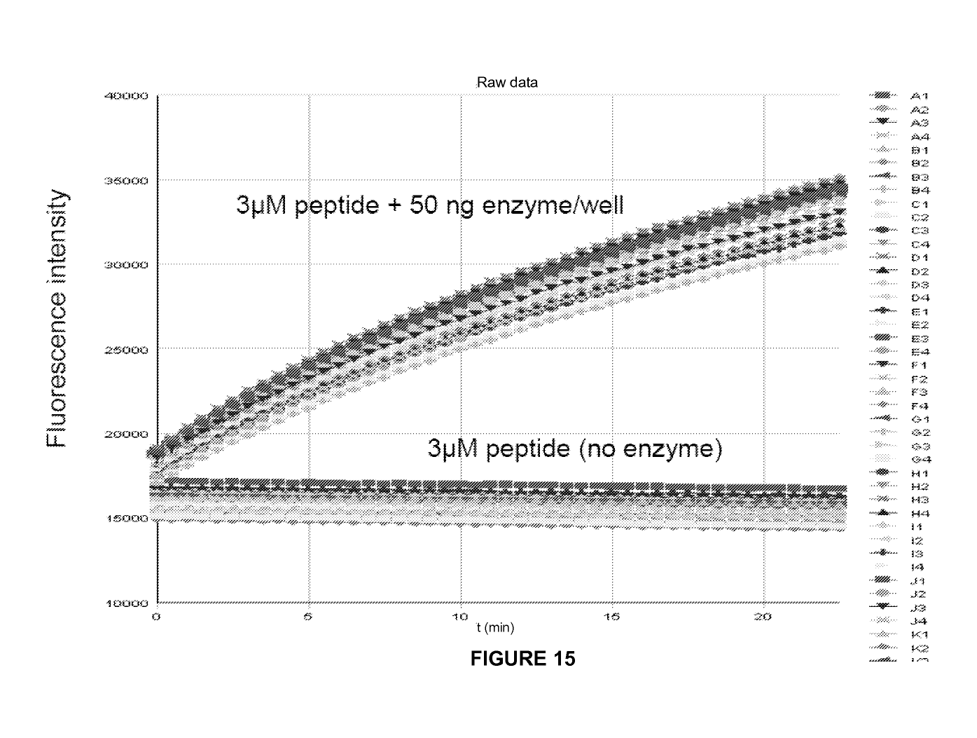

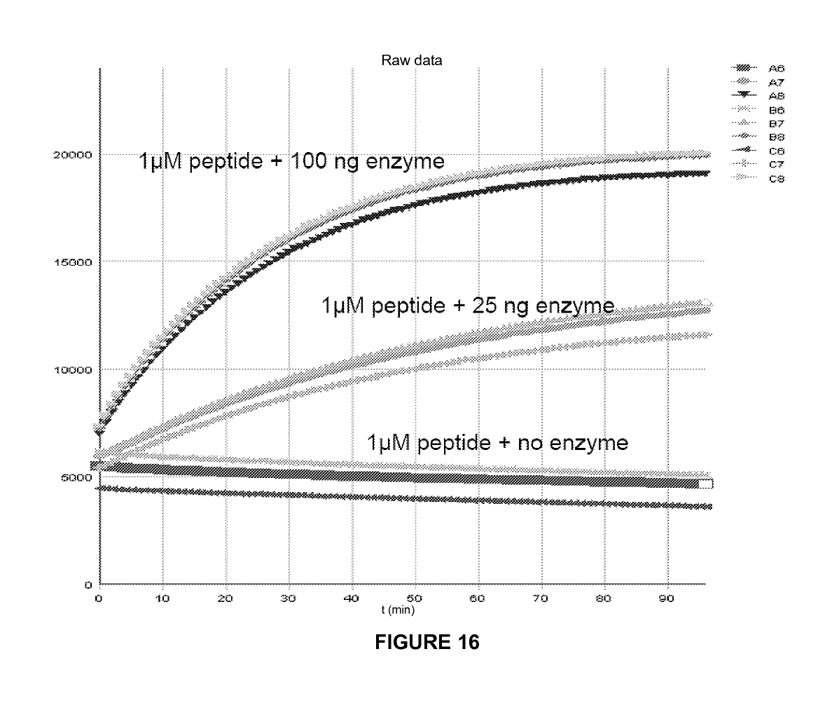

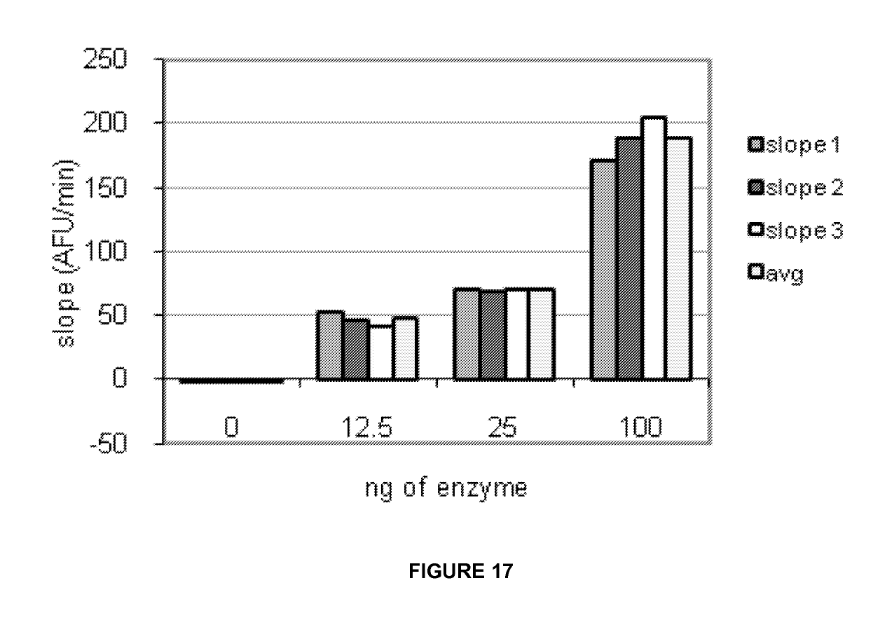

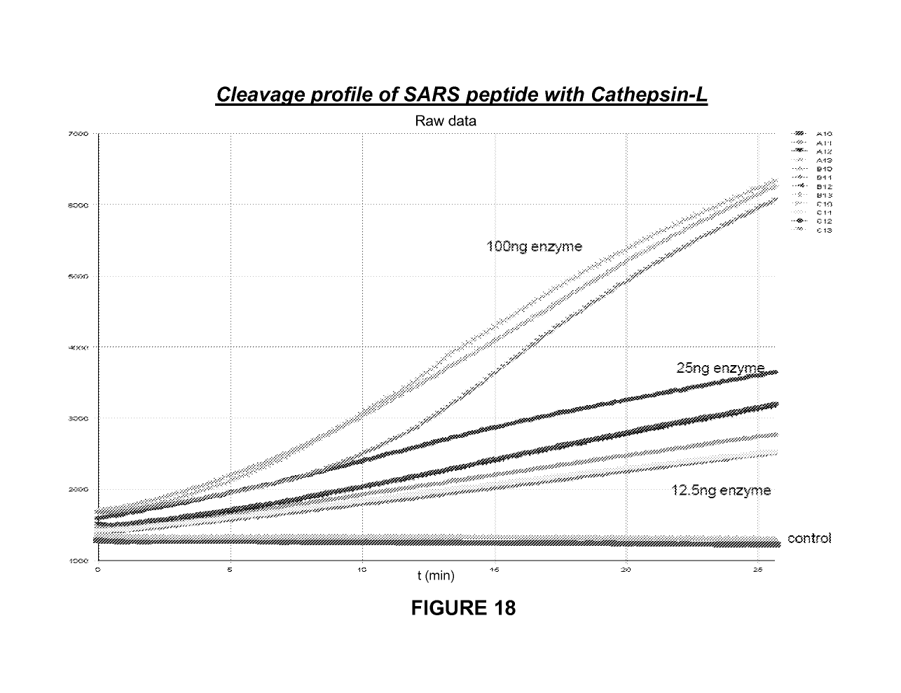

The labeled SARS-CoV derived peptide was cleaved by CatL and the cleavage was measured in the form of increased fluorescence over time with no increase in fluorescence in CatL untreated peptide (FIG. 12A). The different viral peptides showed different rates of cleavage in a dose dependent manner with the host peptides, particularly the host pro-NPY derived peptide, cleaved at a higher rate than the viral derived peptides (FIG. 12B). The validity of the assay was determined based on the Z-factor calculation. Accordingly, the Z-factor was calculated for the different peptides incubated with 0.5 μg/ml CatL after stopping the reaction with 0.5M acetic acid. The Z-factor was found to be between 0.5 and 1 which supports the validity of the HTSA of small molecules that can inhibit the CatL mediated cleavage of the peptide substrates.

### Example 4—HTS of Small Molecules Library Identifies Potential Inhibitors of CatL Mediated Cleavage

Primarily, a library of 5000 small molecules, at 40 μM concentration, from ChemBridge Corporation was tested in duplicates (in black 384 well plates) for inhibition of cathepsin L mediated cleavage of labeled SARS-CoV-S derived peptide in 50 μl total reaction volume using the same buffer as mentioned above. The assay was performed with 1 μM of the peptide incubated with 0.25 μg/ml catL at pH 5.5 for 45 minutes at room temperature after which the reaction was stopped by 10 μl of 0.5M acetic acid. The fluorescence was read using fluorescence reader at the UIC HTS facility. The percentage inhibition of the cathepsin L mediated cleavage by the screened compounds was calculated using the following formula: positive fluorescence signal in absence of compounds−fluorescence signal in presence of compounds×100/positive fluorescence signal in absence of compounds−negative fluorescence signal in absence of the enzyme. The top 50 hits that inhibited the cathepsin L cleavage of SARS-CoV peptide at a cutoff of 60% inhibition were screened in duplicates for the inhibition of cleavage of EBOV-GP, HeV, and NiV-F₀ as well as cleavage of pro-NPY derived labeled peptides as mentioned before.

Fifty compounds, out of the 5000 compounds screened, were identified to inhibit the CatL cleavage of SARS peptide at a cutoff of 60% inhibition calculated based on the fluorescence signal obtained in the absence of the inhibitors (0% inhibition) (Table 1). The validity of the HTSA was further confirmed by calculating the Z-factor using the positive and negative means as well as positive and negative standard deviations. The Z-factor value was found to be 0.61 which confirms our previously calculated Z-factor and ensures that the assay is excellent for HTS of inhibitors of CatL cleavage of the peptide substrates (Table 2).

TABLE 1
Compounds that Inhibit the Cleavage of SARS peptides by Cathepsin−L

| 384 | 384 | 96 | 96 | Read | Read | | | Inh | Pos | Neq | cmpd |
|---|---|---|---|---|---|---|---|---|---|---|---|
| plate# | well# | plate# | well# | A | B | lnh A | lnh B | Mean | Mean | Mean | color |
| 001 | H22 | 90004 | D11 | 459 | 385 | 127.26 | 129.88 | 128.57 | 4049.63 | 1228.11 | black |
| 011 | H03 | 90043 | D02 | 714 | 737 | 119.30 | 117.97 | 118.63 | 2787.31 | 1049.34 | clear |
| 016 | H08 | 90064 | D04 | 878 | 1001 | 110.14 | 104.92 | 107.53 | 3472.27 | 1116.78 | clear |
| 011 | J20 | 90044 | E10 | 896 | 1062 | 108.82 | 99.27 | 104.05 | 2787.31 | 1049.34 | black |
| 011 | N22 | 90044 | G11 | 1030 | 928 | 101.11 | 106.98 | 104.05 | 2787.31 | 1049.34 | clear |
| 011 | N16 | 90044 | G08 | 1065 | 949 | 99.10 | 105.77 | 102.44 | 2787.31 | 1049.34 | orange |
| 011 | N20 | 90044 | G10 | 978 | 1107 | 104.11 | 96.68 | 100.39 | 2787.31 | 1049.34 | orange |
| 008 | K05 | 90029 | F03 | 1580 | 1418 | 89.17 | 93.83 | 91.50 | 4677.05 | 1203.84 | black |
| 016 | N03 | 90063 | G02 | 1480 | 1277 | 84.58 | 93.20 | 88.89 | 3472.27 | 1116.78 | clear |
| 015 | A05 | 90057 | A03 | 1455 | 1352 | 83.93 | 88.55 | 86.24 | 3326.52 | 1096.63 | brown |
| 010 | G18 | 90038 | D09 | 1761 | 1895 | 86.53 | 82.46 | 84.49 | 4612.11 | 1317.11 | clear−light |
| | | | | | | | | | | | orange |
| 014 | N22 | 90056 | G11 | 1350 | 1576 | 89.20 | 78.88 | 84.04 | 3303.42 | 1113.48 | clear |
| 016 | B20 | 90064 | A10 | 1319 | 1732 | 91.41 | 73.88 | 82.65 | 3472.27 | 1116.78 | clear |
| 001 | F14 | 90004 | C07 | 1907 | 1562 | 75.94 | 88.17 | 82.05 | 4049.63 | 1228.11 | dark |
| | | | | | | | | | | | brown |
| 011 | P20 | 90044 | H10 | 1600 | 1230 | 68.32 | 89.61 | 78.96 | 2787.31 | 1049.34 | yellow |
| 013 | D16 | 90052 | B08 | 1403 | 1672 | 85.25 | 72.24 | 78.75 | 3166.19 | 1097.92 | clear |
| 016 | B18 | 90064 | A09 | 1525 | 1802 | 82.67 | 70.91 | 76.79 | 3472.27 | 1116.78 | clear |
| 013 | M14 | 90050 | G07 | 1536 | 1655 | 78.82 | 73.07 | 75.94 | 3166.19 | 1097.92 | pale |
| | | | | | | | | | | | yellow |
| 013 | L14 | 90052 | F07 | 1535 | 1717 | 78.87 | 70.07 | 74.47 | 3166.19 | 1097.92 | clear |
| 010 | N06 | 90040 | G03 | 2296 | 2023 | 70.29 | 78.58 | 74.43 | 4612.11 | 1317.11 | clear |
| 010 | P08 | 90040 | H04 | 2235 | 2168 | 72.14 | 74.18 | 73.16 | 4612.11 | 1317.11 | clear |
| 012 | J21 | 90047 | E11 | 1578 | 1640 | 74.20 | 70.91 | 72.55 | 2974.61 | 1092.41 | clear |
| 016 | G08 | 90062 | D04 | 1444 | 2118 | 86.11 | 57.49 | 71.80 | 3472.27 | 1116.78 | brown |
| 004 | C06 | 90014 | B03 | 1900 | 2100 | 75.27 | 68.27 | 71.77 | 4051.00 | 1193.13 | clear |
| 016 | H22 | 90064 | D11 | 1827 | 1765 | 69.85 | 72.48 | 71.16 | 3472.27 | 1116.78 | clear |
| 010 | P10 | 90040 | H05 | 2150 | 2466 | 74.72 | 65.13 | 69.93 | 4612.11 | 1317.11 | clear−light |
| | | | | | | | | | | | yellow |
| 016 | P20 | 90064 | H10 | 1670 | 2002 | 76.51 | 62.42 | 69.47 | 3472.27 | 1116.78 | clear |
| 011 | L06 | 90044 | F03 | 1659 | 1535 | 64.92 | 72.06 | 68.49 | 2787.31 | 1049.34 | clear |
| 012 | I15 | 90045 | E08 | 1611 | 1778 | 72.45 | 63.57 | 68.01 | 2974.61 | 1092.41 | yellow |
| 007 | E16 | 90026 | C08 | 1991 | 2561 | 76.40 | 59.33 | 67.86 | 4541.59 | 1203.19 | yellow |
| | | | | | | | | | | | brown |
| 014 | B21 | 90055 | A11 | 1663 | 2024 | 74.91 | 58.42 | 66.67 | 3303.42 | 1113.48 | clear |
| 015 | G07 | 90057 | D04 | 1905 | 1808 | 63.75 | 68.10 | 65.92 | 3326.52 | 1096.63 | clear |
| 015 | N17 | 90059 | G09 | 1836 | 1885 | 66.84 | 64.65 | 65.74 | 3326.52 | 1096.63 | pale |
| | | | | | | | | | | | yellow |
| 016 | P16 | 90064 | H08 | 2009 | 1842 | 62.12 | 69.21 | 65.67 | 3472.27 | 1116.78 | clear |
| 016 | P17 | 90063 | H09 | 1836 | 2026 | 69.47 | 61.40 | 65.43 | 3472.27 | 1116.78 | clear |
| 014 | B18 | 90056 | A09 | 2052 | 1693 | 57.14 | 73.54 | 65.34 | 3303.42 | 1113.48 | clear |
| 012 | J12 | 90048 | E06 | 1826 | 1674 | 61.02 | 69.10 | 65.06 | 2974.61 | 1092.41 | clear |
| 004 | L05 | 90015 | F03 | 1918 | 2466 | 74.64 | 55.46 | 65.05 | 4051.00 | 1193.13 | clear |
| 006 | O10 | 90022 | H05 | 1967 | 2123 | 67.17 | 62.24 | 64.70 | 4093.80 | 927.38 | dark |
| | | | | | | | | | | | brown |
| 006 | K13 | 90021 | F07 | 2204 | 1889 | 59.68 | 69.63 | 64.66 | 4093.80 | 927.38 | yellow |
| 008 | B17 | 90031 | A09 | 2532 | 2359 | 61.76 | 66.74 | 64.25 | 4677.05 | 1203.84 | clear |
| 012 | M14 | 90046 | G07 | 1844 | 1691 | 60.07 | 68.20 | 64.13 | 2974.61 | 1092.41 | clear |
| 015 | C17 | 90057 | B09 | 1437 | 2361 | 84.74 | 43.30 | 64.02 | 3326.52 | 1096.63 | clear |
| 008 | K17 | 90029 | F09 | 2496 | 2422 | 62.80 | 64.93 | 63.86 | 4677.05 | 1203.84 | clear |
| 011 | I08 | 90042 | E04 | 1700 | 1688 | 62.56 | 63.25 | 62.91 | 2787.31 | 1049.34 | clear |
| 008 | N13 | 90031 | G07 | 2469 | 2594 | 63.57 | 59.97 | 61.77 | 4677.05 | 1203.84 | dark |
| | | | | | | | | | | | orange |
| 010 | F18 | 90040 | C09 | 2809 | 2360 | 54.72 | 68.35 | 61.54 | 4612.11 | 1317.11 | clear |
| 012 | J06 | 90048 | E03 | 1871 | 1765 | 58.63 | 64.27 | 61.45 | 2974.61 | 1092.41 | clear |
| 015 | H14 | 90060 | D07 | 1812 | 2103 | 67.92 | 54.87 | 61.39 | 3326.52 | 1096.63 | clear |
| 011 | D18 | 90044 | B09 | 1640 | 1801 | 66.01 | 56.75 | 61.38 | 2787.31 | 1049.34 | clear |
| 006 | G20 | 90022 | D10 | 1738 | 2563 | 74.40 | 48.34 | 61.37 | 4093.80 | 927.38 | clear |
| 013 | J18 | 90052 | E09 | 1762 | 2032 | 67.89 | 54.84 | 61.36 | 3166.19 | 1097.92 | clear |
| 011 | L14 | 90044 | F07 | 1673 | 1787 | 64.12 | 57.56 | 60.84 | 2787.31 | 1049.34 | clear |
| 016 | D03 | 90063 | B02 | 1921 | 2173 | 65.86 | 55.16 | 60.51 | 3472.27 | 1116.78 | clear |
| 010 | M18 | 90038 | G09 | 2545 | 2711 | 62.73 | 57.70 | 60.22 | 4612.11 | 1317.11 | clear |

TABLE 2
Assay Statistics

| | | Pos. | | | |
|---|---|---|---|---|---|
| 384plate# | Pos. Mean | STDE | Neg. Mean | Neg. STDE | Z′- factor |
| 001 | 4049.63 | 335.07 | 1228.11 | 126.97 | 0.51 |
| 002 | 3436.08 | 242.79 | 1179.89 | 71.65 | 0.58 |
| 003 | 3793.95 | 240.72 | 1174.05 | 67.27 | 0.65 |
| 004 | 4051.00 | 254.73 | 1193.13 | 79.16 | 0.65 |
| 005 | 3909.66 | 287.65 | 1191.33 | 74.04 | 0.60 |
| 006 | 4093.80 | 323.02 | 927.38 | 154.42 | 0.55 |
| 007 | 4541.59 | 315.72 | 1203.19 | 68.05 | 0.66 |
| 008 | 4677.05 | 294.95 | 1203.84 | 78.51 | 0.68 |
| 009 | 4483.08 | 303.55 | 1328.94 | 82.38 | 0.63 |
| 010 | 4612.11 | 307.07 | 1317.11 | 104.07 | 0.63 |
| average: | 4164.79 | 290.53 | 1194.70 | 90.65 | 0.61 |

### Example 5−Preparation of Pseudotyped Viruses

Pseudotyped viruses (EBOV-GP, SARS-CoV-S, HeV, NiV, and VSV-G) were generated by co-transfection of 2×10⁶ cells of 293FT (grown in DMEM with 10% FBS) with pHIV-GFP-luc expression vector (18 μg), pgagpol HIV vector (1.8 μg) , pHIV-Rev (360 ng) and pHIV-TAT (360 ng) (217), along with the pcDNA3.1-S plasmid (10 μg) coding for the SARS-CoV-S glycoprotein or pcDNA3.1-GP plasmid (10 μg) coding for the EBOV-GP glycoprotein, or pcDNA3.1-VSVG plasmid (1 μg) coding for the VSV-G glycoprotein using calcium phosphate transfection according to the previously described protocol (Coughlin MM et al., 2009, Virology, 10:394 (1): 39-46). For the production of HIV/AE, only HIV vectors were used for transfection. For HeV and NiV pseudotyped viruses, pCAGGs expression plasmids coding for G (15 μg) and F protein (5 μg) of HeV or NiV were transfected along with HIV vectors as described above. The media were changed the following morning and the supernatants were collected 24 and 48 hrs later and pooled. The virus stocks were frozen at −80° C. until used.

### Example 6—HTSA Selected Inhibitors Showed Differential Inhibition to Pseudotyped Virus Entry

The top 50 hits, which showed inhibition of catL cleavage of SARS-CoV derived peptide, were then tested for inhibiting the cleavage of the other viral derived peptides (EBOV, HeV, NiV) and the host pro-NPY derived peptide. (Table 3) Twelve compounds out of 50 showed inhibition to the cleavage of all viral peptides with minimal inhibition of 10% while showing lower inhibition of the pro-NPY derived peptide cleavage (Table 4).

TABLE 3
Assay of Compounds that Inhibit the
Cleavage of SARS, Ebola,
Nipah, Hendra and NPY peptides by Cathepsin-L
A

| | | Ebola | Nipah | Hendra | SARS | NPY |
|---|---|---|---|---|---|---|
| well | compound | peptide | peptide | peptide | peptide | peptide |
| A03 | 6874634 | 4.6 | 46 | 21.2 | 65.6 | 12.8 |
| A04 | 5219666 | 43 | 90.6 | 54.6 | 94.6 | 30 |
| A05 | 5172420 | 6.1 | 55.68 | 8.20 | 42.29 | 4.1 |
| A06 | 7665576 | −3.8 | 63.7 | 37.9 | 60.7 | 2.0 |
| A07 | 7928055 | −16.0 | 18.03 | −0.67 | −0.15 | −0.8 |
| A08 | 5152606 | 31.8 | 121.65 | 108.40 | 82.15 | 70.2 |
| A09 | 7923236 | 14 | 55.1 | 32.6 | 60.2 | 5.2 |
| A10 | 7909513 | −1.6 | 5.15 | 5.24 | 24.06 | 12.0 |
| A11 | 5669125 | −1.8 | 23.57 | −2.70 | 57.39 | 3.3 |
| A12 | 7798500 | −6.3 | 41.5 | 26.3 | 69.6 | 11.2 |
| A13 | 7938158 | −12.6 | 15.92 | −14.40 | 1.34 | −5.3 |
| A14 | 7793889 | −3.4 | 51.1 | −7.77 | 76.5 | 6.1 |
| A15 | 7951692 | 10.2 | 16.58 | 0.34 | 14.51 | 0.7 |
| A16 | 7950181 | −8.6 | 6.45 | −9.26 | 8.55 | −7.0 |
| A17 | 7888659 | 66.9 | 80.96 | 73.49 | 79.93 | 64.8 |
| A18 | 7808526 | 9.6 | 40.8 | −4.08 | 69.2 | 1.2 |
| A19 | 6572698 | 15 | 40.7 | 21.6 | 66.1 | 1 |
| A20 | 5182554 | 31 | 44 | 49.4 | 78.1 | 7.4 |
| A21 | 7924029 | 22 | 50.4 | 26.3 | 60 | 11 |
| A22 | 7914021 | 36 | 53.6 | 25.4 | 71.7 | 13.1 |
| B03 | 7927865 | −3.4 | 27.96 | 0.86 | 52.46 | 6.4 |
| B04 | 7946410 | 5.7 | 18.33 | 6.50 | 24.34 | 8.8 |
| B05 | 7946204 | −24.6 | 12.33 | −7.50 | 39.61 | 9.6 |
| B06 | 5169083 | 13.1 | 49.78 | 35.73 | 44.31 | 29.9 |
| B07 | 5175089 | 6.4 | 2.06 | −2.85 | 38.88 | 10.0 |
| B08 | 7948109 | 6.2 | 46.11 | 37.33 | 38.54 | 14.3 |
| B09 | 7931205 | −0.7 | 66.6 | 34.8 | 36.8 | 19.5 |
| B10 | 7786009 | 2.1 | 58.07 | 37.22 | 51.28 | 10.5 |
| B11 | 7945999 | −0.6 | 55.23 | 33.41 | 39.55 | 4.9 |
| B12 | 7948190 | −2.6 | 45.06 | 7.38 | 33.84 | 11.8 |
| B13 | 7928214 | −6.1 | 12.87 | −0.01 | 23.55 | 0.9 |
| B14 | 5705213 | 42 | 76.3 | 84.3 | 79.8 | 18 |
| B15 | 7910528 | 33.4 | 57.8 | 12.1 | 56.5 | 6.5 |
| B16 | 7976237 | 25.4 | 31.98 | 21.93 | 47.55 | 15.1 |
| B17 | 7927434 | 10.5 | −8.16 | −8.72 | −3.09 | 1.3 |
| B18 | 7948346 | 25.2 | 49.3 | 17.7 | 72.4 | 10.2 |
| B19 | 7914488 | 17.4 | 30.96 | 23.22 | 47.94 | 2.2 |
| B20 | 7557708 | 26.8 | 39.35 | 23.55 | 53.23 | 2.1 |
| B21 | 7927879 | −6.6 | 30.39 | 3.55 | 28.07 | 7.9 |
| B22 | 7947992 | 20 | 61.4 | 31.1 | 37.7 | 18.6 |
| C03 | 7905966 | −12.1 | 9.77 | 3.23 | 37.16 | 6.4 |
| C04 | 7940158 | −12.3 | 34 | 26.2 | 55.8 | 4 |
| C05 | 7790059 | 1.8 | 44.1 | 12.4 | 49.3 | 6.4 |
| C06 | 7760561 | 1.1 | 27.20 | 5.70 | 32.44 | 15.2 |
| C07 | 7945787 | −0.3 | −11.74 | −10.47 | 30.53 | −2.5 |
| C08 | 7963087 | −1.5 | 46.45 | −2.95 | 47.39 | 4.5 |
| C09 | 5728323 | −15.9 | 13.74 | −9.09 | 49.81 | −0.7 |
| C10 | 7946700 | −11.5 | 15.09 | −0.72 | 51.17 | 11.5 |
| C11 | 7924129 | −11.7 | −0.50 | −10.37 | 41.66 | −1.2 |
| C12 | 7944514 | 8.05 | 12.40 | 3.26 | 40.85 | 8.1 |

TABLE 4
Compounds with high percentage inhibition against
viral peptides and low inhibition against human peptide

| Compound | Ebola | Nipah | Hendra | SARS | Pro-NPY |
|---|---|---|---|---|---|
| 6874634 | 48 | 54.1 | 22.7 | 67.7 | 13.4 |
| 5219666 | 75.7 | 87.5 | 65.2 | 96.8 | 44.4 |
| 7923236 | 34.8 | 80.3 | 59 | 66.2 | 10.1 |
| 7798500 | 64.8 | 49.2 | 30.4 | 62.2 | 24.6 |
| 6572698 | 34 | 41.1 | 24.8 | 44.8 | 14.7 |
| 5182554 | 77 | 56.5 | 50.9 | 72.6 | 14.8 |
| 7924029 | 55 | 77.9 | 45.6 | 71.2 | 18.6 |
| 7914021 | 83.8 | 82 | 52.2 | 85.2 | 21.9 |
| 7931205 | 20 | 74.4 | 38.6 | 38.1 | 24.9 |
| 5705213 | 81.3 | 90.7 | 94.2 | 90.1 | 20 |
| 7910528 | 35 | 57.8 | 12.1 | 56.5 | 6.5 |
| 7940158 | 49.1 | 54.7 | 19 | 64.6 | 14.9 |

Specifically, different pseudotyped viruses (EBOV-GP, SARS-CoV-S, and VSV-G as a negative control), normalized for equal infectivity using HIV-1 p24 Elisa kit (Express Biotech International, MD), were mixed with 10 μM of the candidate inhibitory compounds, identified in the screening assay. The virus or virus/compounds mixtures were added to 2×10⁵ 293FT/well seeded in 6 well plates. For the SARS-CoV-S pseudotyped viruses inhibition assays, the 293FT were transiently transfected with human ACE2 expression plasmid (0.4 μg/well), using Effectene transfection reagent (Qiagen) according to the manufacturer's instructions. Twenty four hours later, the cells were transduced with the SARS-CoV-S pseudotyped virus treated or untreated with the candidate compounds. Seventy two hours later, the cells were lysed and the luciferase expression was determined using luciferase assay kit (Promega) according to the manufacturer's instructions.

For HeV and NiV, the compound 5705213 and its derivative 7402683 were used at final concentration of 100 μM during both pseudotyped viruses preparation and transduction. The cathepsin L inhibitor, Z-Phe-Tyr-CHO (Calbiochem, Cat. No. 219402) at 10 μM concentration, was used as a positive control and DMSO treated viruses as a negative control.

The percentage entry inhibition of the candidate compounds on different pseudotyped viruses was calculated using the following formula:

$$\frac{( L ( mock treated virus ) - L ( compound treated virus ) ) \times 100 \%}{L ( mock treated virus ) - L ( HIV Δ E virus )}$$

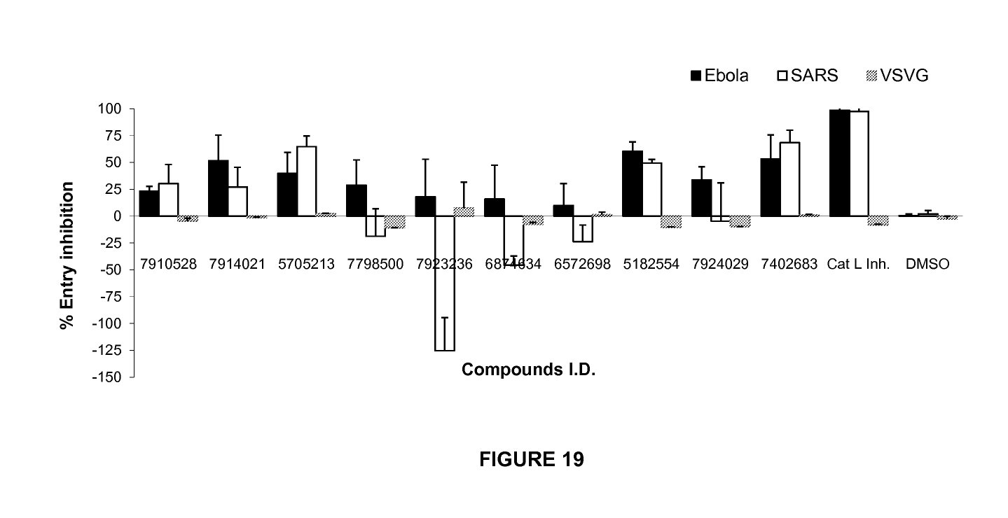

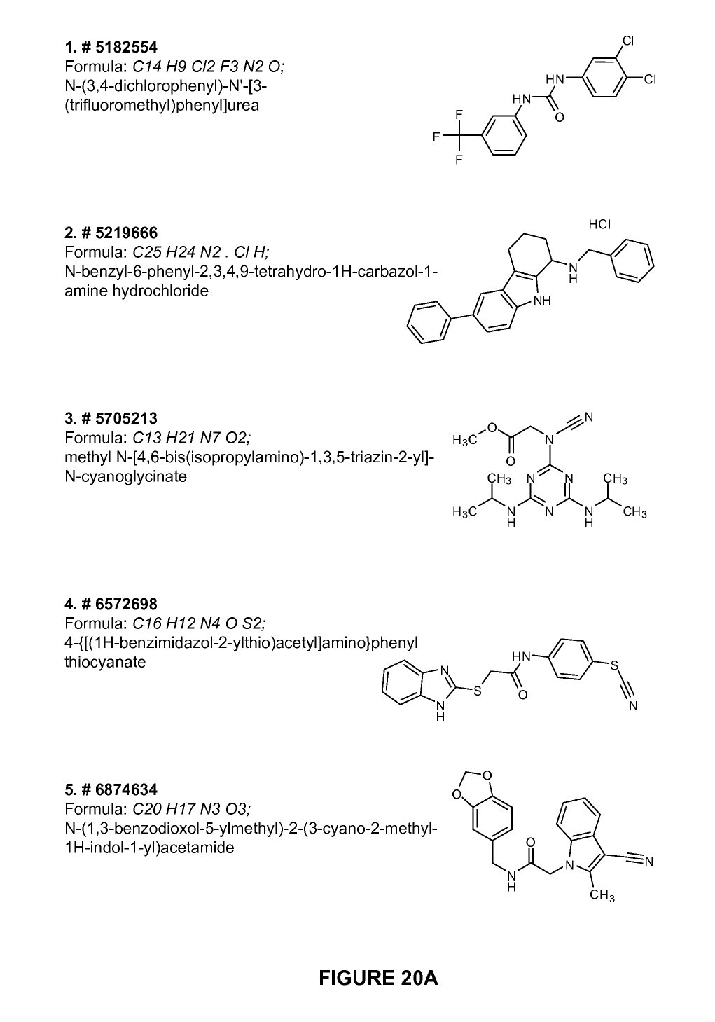

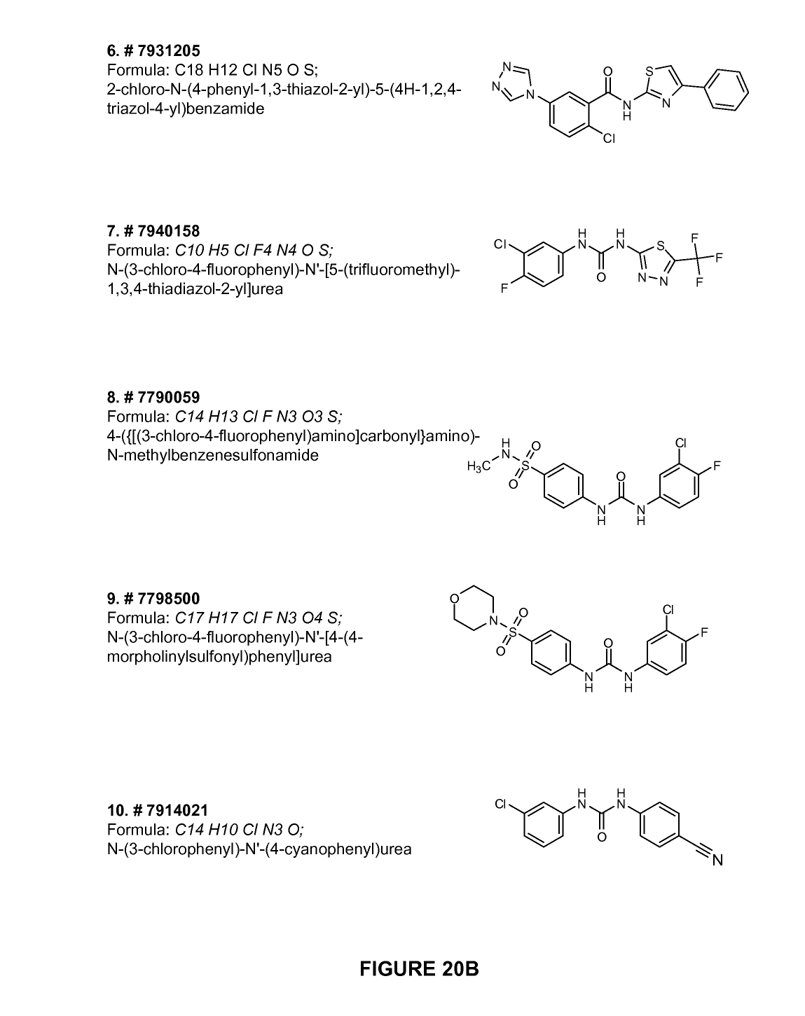

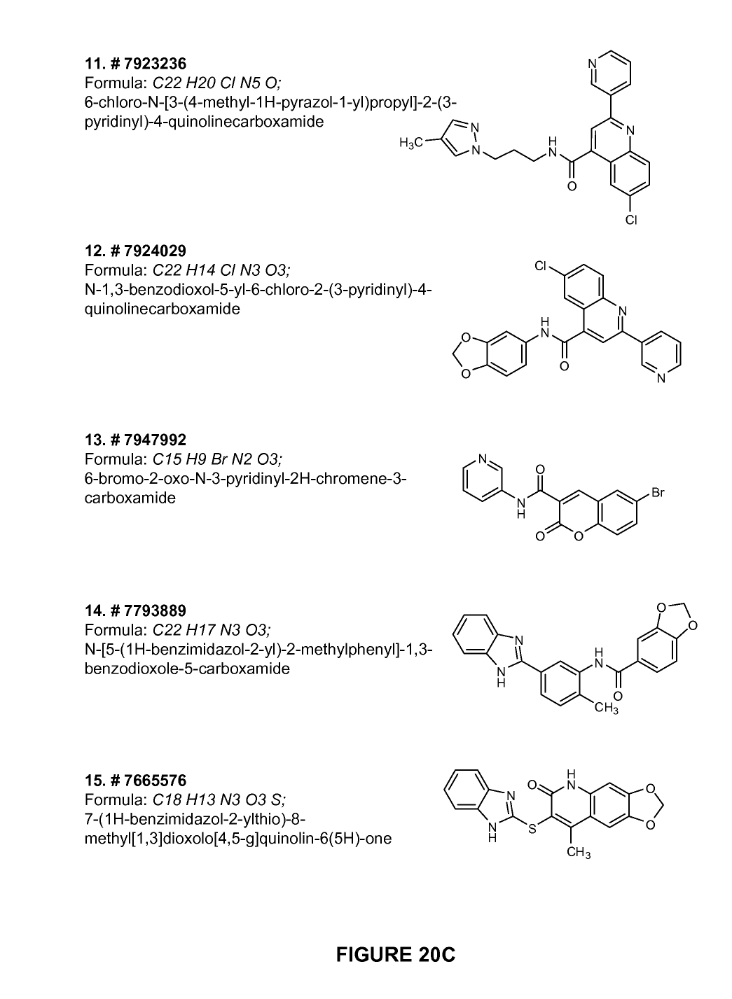

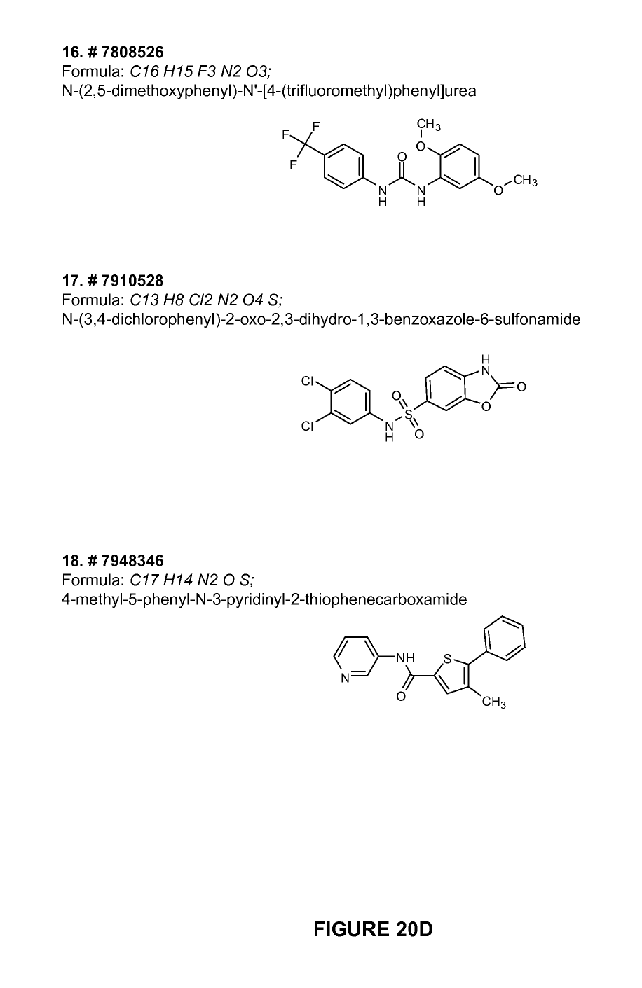

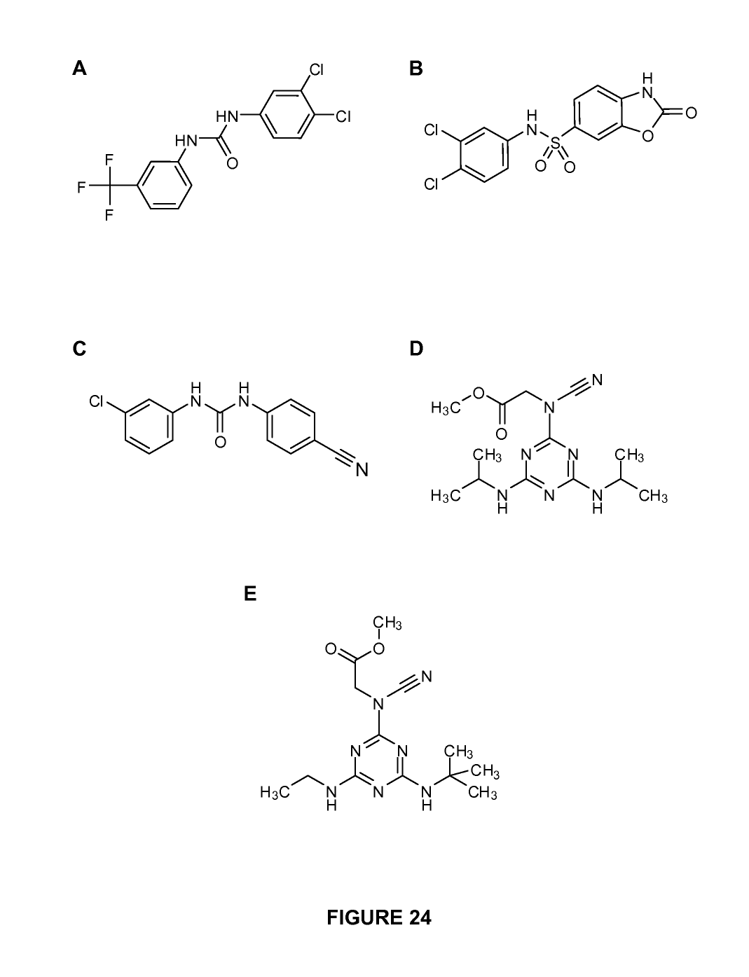

Nine compounds out of 12 identified from the HTSA, that showed higher inhibitions of viral peptide cleavage while minimally inhibiting the pro-NPY derived peptide cleavage, were tested for inhibiting the SARS-CoV-S and EBOV pseudotyped viral entry. Four compounds out of the 9 uncolored compounds were found to inhibit EBOV, and SARS-CoV pseudotyped viruses simultaneously (FIG. 19). These compounds were assigned I.D. numbers by Chembridge Corporation (7910528, 7914021, 5705213, and 5182554) (FIG. 24). The compound 7910528 showed 23.3% and 30.3% inhibitions to EBOV and SARS-CoV pseudotyped viruses respectively. Compound 7914021 showed 51.5% and 27.1% inhibitions to EBOV and SARS-CoV pseudotyped viruses respectively. Compound 5705213 showed 39.8% and 64.7% inhibitions to EBOV and SARS-CoV pseudotyped viruses respectively while compound 5182554 showed 60.45% and 49.3% inhibitions to EBOV and SARS-CoV pseudotyped viruses respectively. Other compounds surprisingly enhanced SARS-CoV-S pseudotyped viral entry. Derivatives of different compounds were tested of which only compound 7402683 showed higher inhibition than its parent compound 5705213 (53.3% and 68.3% inhibitions to EBOV and SARS-CoV-S pseudotyped viruses respectively) (FIG. 19). The DMSO treated cells did not show inhibition of the pseudovirus entry. Similarly, the entry of VSV-G pseudotyped viruses was not affected by any of the compounds. These results show the specificity of the compounds at the indicated concentration towards viruses that utilize the CatL for entry into the target cells.

### Example 7—HTSA Selected Inhibitors Showed Differential Inhibition to Pseudotyped Virus Entry

MTT based cell cytotoxicity assay was performed at different time points to test whether the EBOV and SARS-CoV pseudotyped virus entry inhibition was due to specific effects of the tested compounds or due to an undesirable effect on cell viability and proliferation.

293FT cells were seeded at density of 10⁴ cells/well in 96 well plates. The following day, the cells were treated with different concentrations (10, 30, 50, and 100 μM) of the selected compounds and the cytotoxicity effect of the compounds was assessed using MTT reagent (3-(4,5-Dimethylthiazol-2-yl)-2,5-diphenyltetrazolium bromide) (Roche) for 3 days according to the manufacturer's instructions. Briefly at 24, 48, and 72 hrs following addition of each compound, the cells were washed once with Phosphate buffered Saline (PBS) and fresh DMEM medium without phenol red was added (100 μl/well) after which 10 μl of MTT reagent was added to each well and incubated for 4 hrs at 37° C. 100 μl of 10% SDS solubilized in 0.01M HCl was added to each well with vigorous mixing and further incubated for 4 hrs at 37° C. The OD was measured at 595 nm and the percentage viability was calculated relative to the control after subtracting the background.

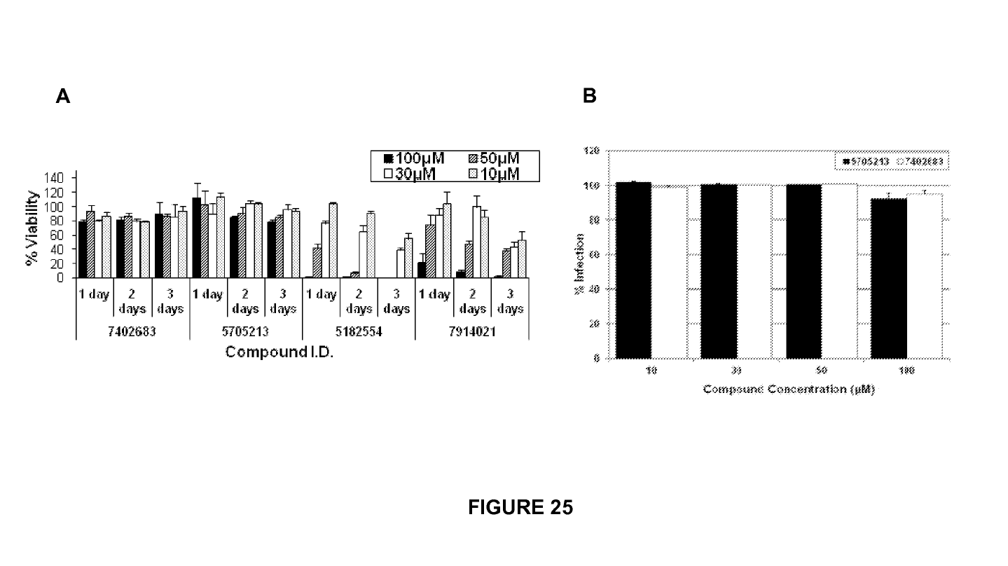

Compounds 7914021 and 5182554 were found to be toxic to the cells at concentrations of 10 μM and above over 3 days of incubation with 293FT cells. The cytotoxic effect was dramatic for the 2 compounds at concentrations above 10 μM after 1 day of incubation with 293FT cells (20-100% cytotoxicity). This cytotoxicity increased over the next 2 days even at 10 μM concentration (FIG. 25A). In contrast, the compound 5705213 did not show a significant cytotoxic effect (cell viability over 80%) on the 293FT cells at 10-100 μM concentration range over 3 days of incubation with 293FT cells (FIG. 25A). Similarly, the derivative 7402683 showed similar pattern with no significant cytotoxicity over 3 days. This confirms the specific inhibitory effect of the compound 5705213 and its derivative on EBOV and SARS-CoV pseudotyped viruses.

To further ensure the specific inhibitory effect of the compounds on the viruses that utilize CatL for entry, the VSV-G pseudotyped virus was treated with different concentrations of the 5705213 compound and its derivative and the luciferase activity was measured 72 hrs post-transduction. Ebola-GP and SARS-CoV-S pseudotyped viruses were mixed with the compounds 5705213 and 7402683 at different concentrations (1-160 μM). The mixture was added to 293FT cells seeded at density of 2×10⁵ cells/well in 6 well plates (In case of SARS-CoV-S pseudotyped viruses, cells were transiently transfected with human ACE2 expression plasmid (pcDNA3.1) 24 hrs prior to transduction). Seventy two hours later, the cells were lysed and the luciferase expression was determined using luciferase assay kit (Promega) according to the manufacturer's instructions. The concentration of each compound was plotted against the percentage inhibition of viral entry and the concentration of the compound that inhibits the viral entry by 50% (IC50) was determined from the curve.

The compounds did not show any significant inhibitory effect on the entry of VSV-G pseudotyped virus even at concentrations up to 100 μM (FIG. 25B). The 5705213 showed an IC50 of 15 μM and 9 μM against EBOV and SARS-CoV-S pseudotyped virus respectively. The derivative showed higher potencies with IC50 of 10 μM and 6 μM against EBOV and SARS-CoV-S pseudotyped virus respectively.

### Example 8—Compound 5705213 and Its Derivative 7402683 Inhibit the in vitro CatL Mediated Cleavage of SARS-CoV-S-Flag Recombinant Protein

To further test the inhibitory effect of the compounds 5705213 and 7402683, SARS-CoV-S-flag recombinant protein was expressed and purified in E. coli BL21 cells.

SARS-CoV-S ectodomain (amino acids 12-1184) was amplified using pcDNA3.1-S expression plasmid expressing full length SARS-CoV-S protein as a template. The forward primer with 5′ 6x His tag-Nhe-I and a reverse primer with 5′ Flag-BamHI were used in the PCR reaction. The PCR product was digested with NheI and BamHI (New England Biolabs) independently and cloned into pet1 11b bacterial expression vector. The recombinant plasmid with His-S-Flag DNA was transformed into DE3 BL21 cells. The transformed cells were induced with 1 mM IPTG at OD=0.8 for 2 hrs after which the cells were lysed with lysis buffer (80 mM Tris-HCl, pH 6.8, 0.006% bromophenol blue, and 15% glycerol). The protein expression was detected by coomassie staining following separation on 4-15% SDS/PAGE gel and confirmed by western blot using monoclonal anti-flag mouse antibody (Sigma Aldrich) and secondary anti-mouse HRP conjugated antibody (Promega). The His-S-Flag inclusion bodies was purified, dissolved in 100 mM Tris-HCl buffer with 8M urea pH=8.8. The protein was further refolded by dilution in refolding buffer (0.1M Tris pH8, 0.4M Arginine, 0.1 mM PMSF, 1 mM EDTA, 5 mM GSH, and 0.5 mM GSSG).

Purified recombinant SARS-CoV-S protein was incubated with 2 μg/ml cathepsin L in NH4 acetate buffer pH=5.5, containing 4 mM EDTA and 8 mM DTT, for 4 hrs in absence and in presence of increasing concentrations of the compound 5705213 and its derivative 7402683. The cleavage of the protein was detected by western blot using anti-flag mouse monoclonal antibody (SigMA Aldrich).

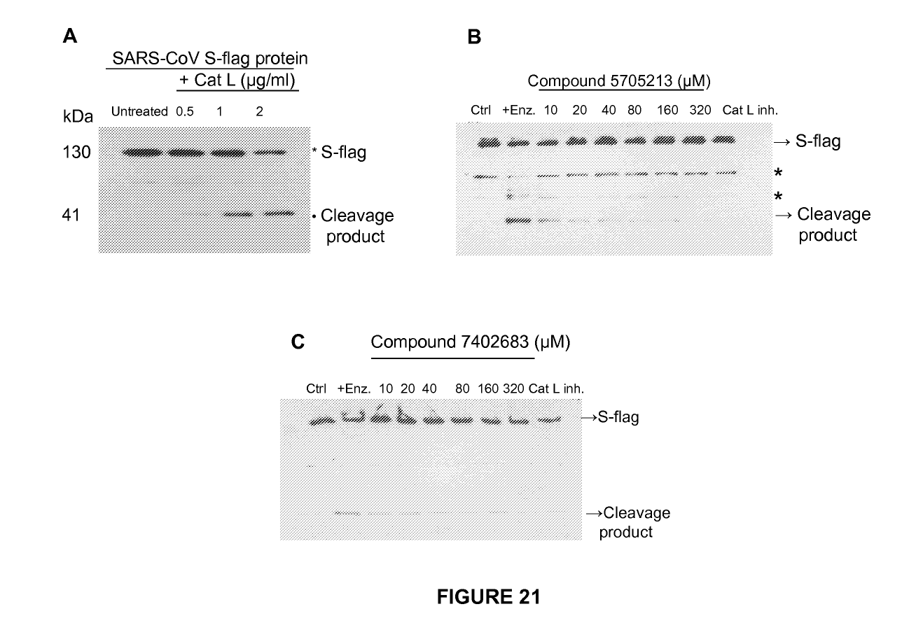

There was a dose dependent cleavage of the recombinant SARS-CoV S-flag protein by Cat L (FIG. 21A). There was a dose dependent inhibition of the Cat L mediated cleavage of SARS-CoV-S-flag protein by the compounds 5705213 (FIG. 21B) and 7402683 (FIG. 21C) with the concentrations tested (10 to 320 μM).

### Example 9—Compound 5705213 and Its Derivative 7402683 Iinhibit the Endogenous Processing of Nipah and Hendra Fusion Proteins and the Entry of Pseudotyped Viruses

Next, the inhibitory effect of the compound 5705213 and its derivative 7402683 was tested on the endogenous processing of NiV and HeV F₀ proteins in 293FT cells transiently expressing the fusion proteins.

293FT cells were plated at a density of 200,000 cells/well in 6 well plates. The cells were transfected with Nipah or Hendra virus F₀ expression plasmid (2 μm) using polyfect transfection reagent (Qiagen) then treated with the inhibitors (5705213 and its derivative 7402683) at 100 μM concentration 4 hrs post-transfection. The cathepsin L inhibitor was used as a control at 10 μM concentration. The cells were lysed 48 hrs later and F₀ processing was determined by western blot using cross reactive anti-Nipah and Hendra F protein monoclonal antibody. F₀ represents uncleaved fusion protein while F₁ is the fusion subunit of the F₀ protein.

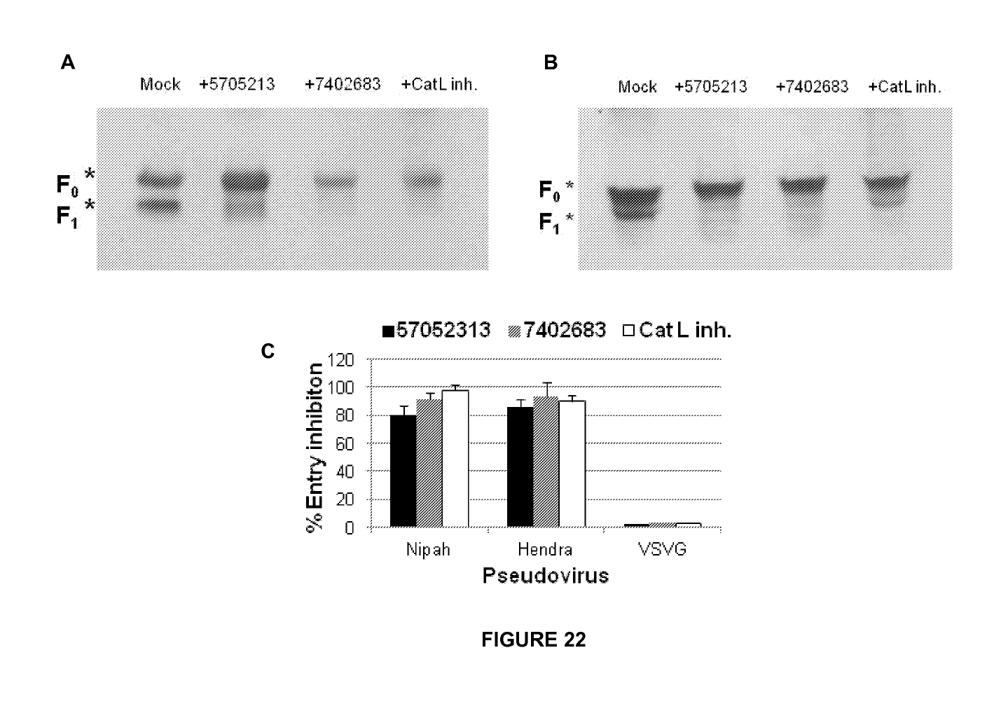

Both compounds were able to efficiently inhibit the endogenous cleavage by CatL when compared to the inhibition seen with the commercial cathepsin L inhibitor (FIGS. 22A & 22B). The two compounds as well as the control (cathepsin L inhibitor) inhibited the entry of HeV and NiV pseudotyped viruses into 293FT cells with entry inhibitions of 80 to 100% (FIG. 22C). As expected, the VSV-G pseudotyped virus entry was unaffected by the compounds (FIG. 22C).

### Example 10—SARS-CoV-S Pseudotyped Virus Entry Inhibition Is More Dramatic By a Combination of the Cmpound 5705213 and the Protease Inhibitor In Cells Expressing TMPRSS2 Protease

SARS-CoV natural infection of pneumocytes can be activated by transmembrane protease/serine subfamily member 2 (TMPRSS2) expressed on the cell surface or CatL in the late endosomes (Simmons G, et al., 2005, Proc Natl Acad Sci USA 102:11876-81; Shulla A, et al., 2011, J Virol. 85(2):873-82; Matsuyama S, et al., 2010, J Virol. 84(24):12658-64). Therefore, the inhibition of SARS-CoV infection would be optimum when using a combination of protease inhibitor and CatL inhibitor. Thus, the entry of the SARS-CoV-S pseudotyped virus into 293FT expressing either the receptor (ACE2) or the receptor plus different amounts of the TMPRSS2 was tested.

293FT cells were plated at a density of 200,000 cells/well in 6 well plates. The 293FT cells were transfected with the human SARS-CoV receptor ACE2 plasmid only (1 μg) or the receptor plus increasing amounts of TMPRSS2 protease plasmid (10-800 ng) using polyfect transfection reagent (Qiagen). The cells were transduced with the SARS-CoV pseudotyped virus, quantified using HIV-1 p24 Elisa kit (Express Biotech International, MD), 24 hrs post-transfection. HIV/AE virus which does not express the SARS-CoV S protein was used as a negative control of entry. The entry was quantified by measuring the luciferase expression in cell lysates 72 hrs post-transduction in terms of Relative Light Units (RLU). The entry of pseudotyped SARS-CoV into 293FT cells, transfected with either the receptor only (ACE2 plasmid) or receptor plus 10 ng of TMPRSS2 plasmid, was measured in presence or absence (mock) of different inhibitors. Camostat mesylate was used as protease inhibitor (Tocris Bioscience) and Z-Phe-Tyr-CHO as a Cathepsin L inhibitor (Calbiochem, Cat.No. 219402). All the inhibitors were used at 10 μM concentration.

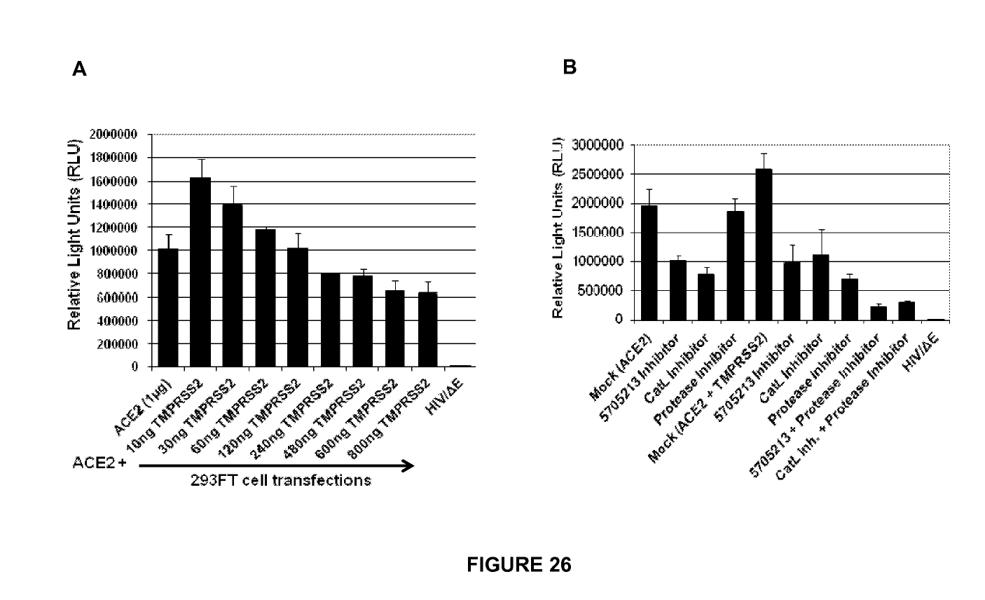

There was a significant increase in pseudotyped virus entry in cells transfected with 10 ng of the TMPRSS2 plasmid, relative to the entry in cells with no TMPRSS2 expression, which decreases upon increasing the amount of the TMPRSS2 plasmid transfected (FIG. 26A). This may be due to unspecific proteolytic effects of the high concentration of the TMPRSS2 protease.

Next, the inhibitory effect of the compound 5705213, the protease inhibitor (Camostat) and a combination of both in 293FT cells expressing the SARS-CoV receptor, human ACE2, with or without the expression of the membrane protease TMPRSS2 (10 ng) was tested. The compound 5705213 and the commercial CatL inhibitor unlike the protease inhibitor were able to inhibit the entry by 50 and 60% respectively in absence of the TMPRSS2. The inhibition was found to be 60%, 58%, 70%, 95%, and 90% for 5705213, CatL inhibitor, the protease inhibitor, the combination of 5705213+protease inhibitor, and the combination of CatL inhibitor+protease inhibitor respectively (FIG. 26B). This suggests that the identified inhibitor 5705213 may be used in combination with the commercial protease inhibitor to completely block SARS-CoV natural lung infection.

### Example 11—Compound 5705213 is a Mixed Inhibitor For Cathepsin L

In order to determine the mechanism of action of the identified inhibitor, an enzyme kinetics assay based on Michaelis-Menten kinetics was performed.

Different concentrations of the SARS-CoV-S, EBOV-GP, HeV-F₀, and NiV-F₀ derived labeled peptide (2-64 μM) were incubated with fixed concentration of cathepsin L (0.5 μg/ml) in absence or presence of 5705213 compound for 40 minutes at room temperature. The reaction was stopped with 10 μl of 0.5M acetic acid after which the fluorescence was read, at 535 nm after excitation at 485 nm, with the fluorescence reader at UIC HTS facility. The reaction was performed in 50 μl total volume in 384 well plates using NH₄ acetate buffer pH5.5 supplemented with 4 mM EDTA and 8 mM DTT. The Velocity of the reaction at different substrate concentrations was calculated (Fluorescence units/minute) and plotted versus substrate concentration. The inverse velocity was further plotted versus inverse substrate concentration (Lineweaver-Burk plot) from which the Kₘ and Vₘₐₓ were calculated.

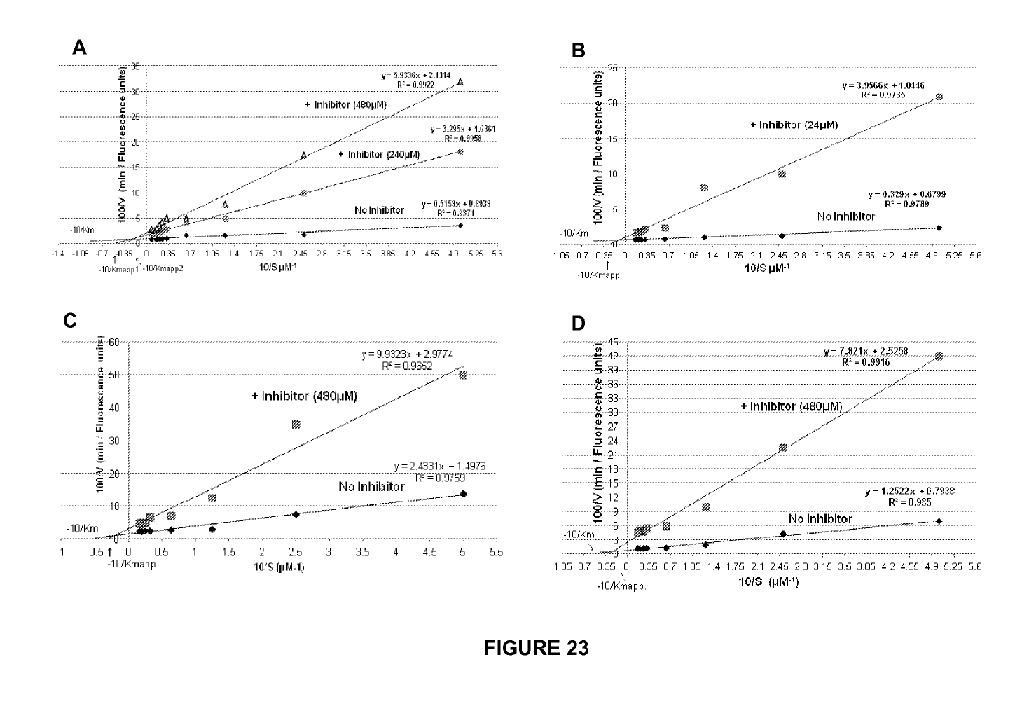

Lineweaver-Burk plot provides a graphical way for analysis of the Michaelis-Menten equation: V=Vₘₐₓ [S]/Kₘ+[S], taking the reciprocal gives 1/V=Kₘ+[S]/Vₘₐₓ [S]=Kₘ/Vₘₐₓ·1/[S]+1/Vₘₐₓ which is the equation for the Lineweaver-Burk plot, where V is the reaction velocity (the reaction rate), Kₘ is the Michaelis-Menten constant, Vₘₐₓ is the maximum reaction velocity, [S] is the substrate concentration, and Kₘ/Vₘₐₓ is the slope. From the Lineweaver-Burk plot, it was found that there was an increase of Kₘ and a decrease in Vₘₐₓ in presence of the inhibitor compound 5705213 whicICh is dose dependent (FIG. 23). This suggested that the identified compound is a mixed inhibitor based on currently established kinetics.

Although certain presently preferred embodiments of the disclosure have been described herein, it will be apparent to those of skill in the art to which the disclosure pertains that variations and modifications of the described embodiment may be made without departing from the spirit and scope of the disclosure. Accordingly, it is intended that the disclosure be limited only to the extent required by the following claims and the applicable rules of law.

We claim:

1. A method of inhibiting a viral infection in a mammal in need thereof, the viral infection being caused by a virus which requires membrane fusion for viral entry, the method comprising administering to the mammal an effective amount of a compound having the structural formula (I): 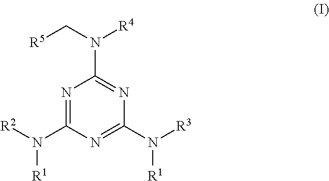
or a pharmaceutically acceptable salt thereof, wherein
each R¹ is independently hydrogen or C₁-C₆ alkyl;
R² is C₁-C₆ alkyl, C₁-C₆ haloalkyl, C₃-C₈ cycloalkyl-, heterocyclyl-, aryl-, heteroaryl- or —C₁-C₆ alkyl-R⁶, each optionally substituted by one or more groups that are each independently halogen, cyano, nitro, hydroxy, C₁-C₆ alkoxy, amino, (C₁-C₆ alkyl)amino, or di(C₁-C₆ alkyl)amino;
R³ is C₁-C₆ alkyl, C₁-C₆ haloalkyl, C₃-C₈ cycloalkyl-, heterocyclyl-, aryl-, heteroaryl- or —C₁-C₆ alkyl-R⁶, each optionally substituted by one or more groups that are each independently halogen, cyano, nitro, hydroxy, C₁-C₆ alkoxy, amino, (C₁-C₆ alkyl)amino, or di(C₁-C₆ alkyl)amino;
R⁴ is C₁-C₆ alkyl, C₁-C₆ haloalkyl, —CN, —C(O)C₁-C₆ alkyl, —C(O)C₁-C₆ alkoxy, —C(O)NR⁹R⁹, or —S(O)₂C₁-C₆ alkyl; and
R⁵ is hydroxy, C₁-C₆ alkoxy, amino, (C₁-C₆ alkyl)amino, di(C₁-C₆ alkyl)amino, —C(O)C₁-C₆ alkyl, —C(O)C₁-C₆ haloalkyl, —C(O)O—C₁-C₆ alkyl, —C(O)O—C₁-C₆ haloalkyl, or —C(O)NR⁹R⁹;
wherein each R⁶ is independently selected from the group consisting of: —OR⁷, —SR⁷, —NR⁸R⁸, —C(O)R⁷, —C(O)OR⁷, —C(O)NR⁸R⁸, —S(O)₂NR⁸R⁸, —OC(O)R⁷, —N(R⁷)C (O)R⁷, —OC(O)OR⁷, —OC(O)NR⁸R⁸, —N(R⁷)C(O)OR⁷, —N(R⁷)C(O)NR⁸R⁸, and —N(R⁷)S(O)₂R⁷;
and each R⁷ is independently hydrogen, C₁-C₆ alkyl, or C₁-C₆ haloalkyl;
each R⁸ is independently hydrogen or C₁-C₆ alkyl; and
each R⁹ is independently hydrogen or C₁-C₆ alkyl.

2. A method of claim 1, wherein each R¹ is hydrogen.

3. A method of claim 1 or 2, wherein R² is C₁-C₆ alkyl or —C₁-C₆ alkyl-R⁶, wherein R⁶ is —OR⁷, —SR⁷, or —NR⁸R⁸.

4. A method of any one of claims 1-3, wherein R³ is C₁-C₆ alkyl or —C₁-C₆ alkyl-R⁶, wherein R⁶ is —OR⁷, —SR⁷, or —NR⁸R⁸.

5. A method of any one of claims 1-4, wherein R² and R³ are independently selected from ethyl, i-propyl, and t-butyl.

6. A method of any one of claims 1-5, wherein R⁴ is —CN.

7. A method of any one of claims 1-6, wherein R⁵ is —C(O)C₁-C₆ alkyl, —C(O)C₁-C₆ haloalkyl, —C(O)C₁-C₆ alkoxy, or —C(O)C₁-C₆ haloalkoxy.

8. A method of claim 7, wherein R⁵ is —C(O)₂CH₃.

9. A method according to claim 1, wherein the compound is:
methyl 2-(N-(4,6-bis(isopropylamino)-1,3,5-triazin-2-yl)cyanamido)acetate; or
methyl 2-(N-(4-(tert-butylamino)-6-(ethylamino)-1,3,5-triazin-2-yl)cyanamido)acetate.

10. The method according to any one of claims 1-9 wherein the virus is selected from the group consisting of: severe acute respiratory syndrome coronavirus, Ebola virus, Hendra virus, and Nipah Virus.

11. A method of inhibiting a viral infection in a mammal in need thereof, the viral infection being caused by a virus which requires membrane fusion for viral entry, the method comprising administering to the mammal an effective amount of a compound having structural formula (II): 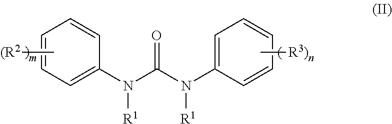
or a pharmaceutically acceptable salt thereof, wherein
m is an integer 0, 1, 2, or 3;
n is an integer 0, 1, 2, or 3;
each R¹ is independently hydrogen or C₁-C₆ alkyl;
each R² is independently halogen, —CN, —NO₂, —N₃, C₁-C₆ alkyl, —C₁-C₆ haloalkyl, —R⁴, or —C₁-C₆ alkyl-R⁴; and
each R³ is independently halogen, —CN, —NO₂, —N₃, C₁-C₆ alkyl, —C₁-C₆ haloalkyl, —R⁴, or —C₁-C₆ alkyl-R⁴,
wherein each R⁴ is independently selected from the group consisting of: —OR⁵, —SR⁵, —S(O)R⁵, —S(O)₂R⁵, —NR⁶R⁶, —C(O)R⁵, —C(O)OR⁵, —C(O)NR⁶R⁶, —S(O)₂NR⁶R⁶, —OC(O)R⁵, —N(R⁵)C(O)R⁵, and —N(R⁵)S(O)₂R⁵, in which each R⁵ is independently hydrogen, C₁-C₆ alkyl, or C₁-C₆ haloalkyl, and each R⁶ is independently hydrogen or C₁-C₆ alkyl.

12. A method of claim 11, wherein each R¹ is hydrogen.

13. A method of claim 11 or 12, wherein m is integer 2.

14. A method of any one of claims 11-13, wherein n is an integer 1.

15. A method according to any one of claims 11-14, wherein each R² and each R³ are independently halogen, —CN, or C₁-C₆ haloalkyl.

16. A method according to claim 15, wherein each R² is independently halogen, and each R³ is C₁-C₆ haloalkyl.

17. A method according to claim 15, wherein each R² is independently halogen, and each R³ is —CN.

18. A method according to claim 11, wherein the compound is:
1-(3,4-dichlorophenyl)-3-(3-(trifluoromethyl)phenyl)urea; or
1-(4-chlorophenyl)-3-(4-cyanophenyl)urea.

19. The method according to any one of claims 11-18 wherein the virus is selected from the group consisting of: severe acute respiratory syndrome coronavirus, Ebola virus, Hendra virus, and Nipah Virus.

20. A method of inhibiting viral infection in a mammal in need thereof, the viral infection being caused by a virus which requires membrane fusion for viral entry, the method comprising administering to the mammal an effective amount of a compound having structural formula (III): 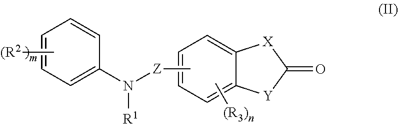
or a pharmaceutically acceptable salt thereof, wherein
X is —CH—, —NR⁷—, —O—, or —S—, where R⁷ is hydrogen or C₁-C₆ alkyl;
Y is —CH—, —NR⁸, —O—, or —S—, where R⁸ is hydrogen or C₁-C₆ alkyl;
Z is —S(O)₂— or —C(O)—;
m is an integer 0, 1, 2, 3, or 4;
n is an integer 0, 1, 2, or 3;
each R¹ is independently hydrogen or C₁-C₆ alkyl;
each R² is independently halogen, —CN, —NO₂, —N₃, C₁-C₆ alkyl, C₁-C₆ haloalkyl, —R⁴, or —C₁-C₆ alkyl-R⁴; and
each R³ is independently halogen, —CN, —NO₂, —N₃, C₁-C₆ alkyl, C₁-C₆ haloalkyl, —R⁴, or —C₁-C₆ alkyl-R⁴,
wherein each R⁴ is independently selected from the group consisting of: —OR⁵, —SR⁵, —S(O)R⁵, —S(O)₂R⁵, —NR⁶R⁶, —C(O)R⁵, —C(O)OR⁵, —C(O)NR⁶R⁶, —S(O)₂NR⁶R⁶, —OC(O)R⁵, —N(R⁵)C(O)R⁵, —OC(O)OR⁵, —OC(O)NR⁶R⁶, —N(R⁵)C(O)OR⁵, —N(R⁵)C(O)NR⁶R⁶, and —N(R⁵)S(O)₂R⁵, in which each R⁵ is independently hydrogen, C₁-C₆ alkyl, or C₁-C₆ haloalkyl, and each R⁶ is independently hydrogen or C₁-C₆ alkyl.

21. A method of claim 20, wherein each R¹ is independently hydrogen, m is an integer 2, and each R² is independently halogen, —CN, or C₁-C₆ haloalkyl.

22. A method according to claim 21, wherein each R² is independently halogen.

23. A method of any one of claims 20-22, wherein Z is —S(O)₂—.

24. A method of any one of claims 20-23, wherein one of X or Y is —NR¹—, and the other is —O—.

25. A method according to claim 20, wherein the compound is:
N-(3,4-dichlorophenyl)-2-oxo-2,3-dihydrobenzo[d]oxazole-6-sulfonamide.

26. The method according to any one of claims 20-25, wherein the virus is selected from the group consisting of: severe acute respiratory syndrome coronavirus, Ebola virus, Hendra virus, and Nipah Virus.

27. A method according to any of claims 1-26, wherein the virus is severe acute respiratory syndrome coronavirus.

28. A method according to any of claims 1-26, wherein the virus is Ebola virus.

29. A method according to any of claims 1-26, wherein the virus is Hendra virus.

30. A method according to any of claims 1-26, wherein the virus is Nipah Virus.

31. A method for inhibiting cathepsin L-mediated cleavage of viral glycoprotein-derived peptide in a virus, the method comprising contacting the virus with an effective amount of a compound or pharmaceutically acceptable salt as described in any of claims 1-26.

32. A method according to claim 31, wherein the virus is selected from the group consisting of severe acute respiratory syndrome coronavirus, Ebola virus, Hendra virus and Nipah Virus.

33. A pharmaceutical composition comprising a therapeutically effective amount of a compound or pharmaceutically acceptable salt according to any of claims 1-26, and one or more pharmaceutically acceptable diluents, preservatives, solubilizers, emulsifiers, adjuvants, excipients, or carriers.
